(19) United States
(12) Patent Application Publication
(10) Pub. No.: US 2022/0119772 A1
(43) Pub. Date: Apr. 21, 2022

(54) COMPOSITIONS AND METHODS FOR TREATMENT OF A MALABSORPTIVE DISORDER

(71) Applicant: CHILDREN'S HOSPITAL MEDICAL CENTER, Cincinnati, OH (US)

(72) Inventors: Heather McCauley, Cincinnati, OH (US); James Macormack Wells, Cincinnati, OH (US)

(21) Appl. No.: 17/421,908

(22) PCT Filed: Jan. 14, 2020
(86) PCT No.: PCT/US2020/013428

§ 371 Date: Jul. 9, 2021

Related U.S. Application Data
(60) Provisional application No. 62/791,937, filed on Jan. 14, 2019.

(51) Int. Cl. C12N 5/071; G01N 33/50

(52) U.S. Cl. CPC C12N 5/0679; G01N 33/5044; C12N 2533/90; C12N 2513/00; C12N 2506/02

(57) ABSTRACT

Described herein are methods for treating a malabsorptive disorder. The malabsorptive disorder may be, in certain aspects, characterized by malabsorption of macronutrients in the intestine, and may include, for example, a disease selected from one or more of enteric anendocrinosis, short gut syndrome, enteric pathogen infection, malnutrition, genetic causes of malabsorption, Celiac disease, malabsorptive diarrhea, and inflammatory bowel. Such methods may include administration of peptide YY (PYY) to an individual in need thereof. Also described are medicaments for carrying out the disclosed methods.

### CROSS-REFERENCE TO RELATED APPLICATIONS

This application claims priority to and benefit of U.S. Provisional Application Ser. No. 62/791,937 filed Jan. 14, 2019, entitled Enteroendocrine cells couple an epithelial-neuronal signal to control nutrient absorption,” the contents of which are incorporated in their entirety for all purposes.

### STATEMENT REGARDING FEDERALLY-SPONSORED RESEARCH

This invention was made with government support under DK092456 awarded by the National Institutes of Health. The government has certain rights in the invention.

### BACKGROUND

The ability to absorb ingested nutrients and/or micronutrients is an essential function of all metazoans and utilizes a wide array of nutrient transporters found on the absorptive enterocytes of the small intestine. In certain disease states, the absorptive capacity of an individual may be compromised, leading to disordered absorption of nutrients, and in some instances, malabsorptive diarrhea, or metabolic acidosis, thus requiring parenteral nutrition or small bowel transplant for survival. Poor absorption of macronutrients is a global health concern, with underlying etiology including short-gut syndrome, enteric pathogen infection, and malnutrition. In certain other instances, modulating the absorption of ingested nutrients and/or micronutrients may be advantageous in certain disease states such as obesity. In such instances, it may be beneficial to reduce nutrient absorption in an individual having excess weight or obesity. The instant disclosure seeks to address one or more of the aforementioned needs in the art.

### BRIEF SUMMARY

Described herein are methods for treating a malabsorptive disorder. The malabsorptive disorder may be, in certain aspects, characterized by malabsorption of macronutrients in the intestine, and may include, for example, a disease selected from one or more of enteric anendocrinosis, short gut syndrome, enteric pathogen infection, malnutrition, genetic causes of malabsorption, Celiac disease, malabsorptive diarrhea, and inflammatory bowel. Such methods may include administration of peptide YY (PYY) to an individual in need thereof. Also described are medicaments for carrying out the disclosed methods.

### BRIEF DESCRIPTION OF THE DRAWINGS

This application file contains at least one drawing executed in color. Copies of this patent or patent application publication with color drawing(s) will be provided by the Office upon request and payment of the necessary fee.

Those of skill in the art will understand that the drawings, described below, are for illustrative purposes only. The drawings are not intended to limit the scope of the present teachings in any way.

FIG. 1A-1D. Ion transport is deranged in EEC-deficient mouse and human small intestine. 1A. EEC-deficient enteroids have heightened response to VIP. Addition of VIP to enteroids induced ion and water transport as measured by organoid swelling. EEC-deficient enteroids (n=45) had an elevated response to VIP compared to wild type enteroids (n=43) (*p=0.04). Upon addition of PYY, there was no difference in swelling between wild type (n=30) and EEC-deficient enteroids (n=70), and significant inhibition of VIP-induced swelling (wild type, ***p=4e-9; mutant, ***p=1e-10). VIP-induced enteroid swelling was CFTR dependent and blocked by the CFTR inhibitor CFTR-172 (n=30 wild type, n=30 EEC-deficient). Scale bars=500 μm. Black bars represent wild type and gray bars represent EEC-deficient enteroids. Error bars are±s.e.m.; statistics calculated by unpaired, two-tailed Student's t-test. 1B. EEC-deficient enteroids displayed impaired NHE3 activity. EEC-deficient enteroids exhibited reduced Nat-dependent recovery of intracellular pH after an acid load using the ratiometric pH indicator SNARF-4F. Quantification is of initial rate of Nat-dependent pH recovery (red line). n=16 wild-type, n=23 mutant enteroids; *p=0.04. Error bars are±s.e.m.; statistics calculated by unpaired, two-tailed Student's t-test. 1C. The levels and localization of the VIP receptor VIPR1 and PYY receptor NPY1R are comparable between wild type and EEC-deficient human intestinal epithelium. PYY+ and CHGA+ cells were only found in wild-type HIOs. Scale bars=100 μm. 1D. EEC-deficient human and mouse small intestinal tissues have a deranged electrochemical response to VIP that can be normalized with PYY. In the Ussing chamber, EEC-deficient small intestine displayed a greater response to 10 nM VIP than did wild-type (mouse, n=20 wild-type, 8 mutant, ***p=0.0003; human, n=16 wild-type, 9 mutant, **p=0.006). Addition of exogenous PYY reduced the magnitude of response (ΔI_{sc}) to VIP (n=8 mutant mice, **p=0.003 from untreated; n=7 mutant HIOs, *p=0.01 from untreated) to wild-type levels. Inhibition of the PYY receptor in wild-type tissue with BIB03304 resulted in an elevated response to VIP compared to untreated wild-type (mouse, n=24, *p=0.03 from untreated; human, n=7, *p=0.04 from untreated). All electrogenic responses to VIP were blocked by the CFTR inhibitor CFTR-172. Dotted lines represent wild type (black) and mutant (gray) jejunum pre-treated with 20 μM CFTR-172. One representative trace is shown (mouse), with baseline I_{sc} was normalized to 0 μA/cm². Error bars are±s.e.m.; statistics calculated by unpaired, two-tailed Student's t-test.

FIG. 2A-2G. PYY restores normal glucose absorption in EEC-deficient human and mouse small intestine. 2A. Schematic depicting the PYY-VIP paracrine axis regulating ion and water homeostasis. EEC-derived PYY and ENS-derived VIP both act via G-protein coupled receptors (NPY1R and VIPR1, respectively) on enterocytes. VIP signaling raises intracellular cAMP levels resulting in activation of CFTR and efflux of chloride ions while concurrently inhibiting the sodium-hydrogen exchanger NHE3. The downstream results are that water and sodium are drawn to the intestinal lumen via paracellular spaces to balance the secreted chloride. PYY is secreted in response to luminal nutrients and acts as a counterbalance to VIP by lowering intracellular cAMP levels. Transport of luminal nutrients into the enterocyte depends on these ion gradients; S GLT1 transports glucose with two Na+ ions and PEPT1 transports di-/tri-peptides with an H⁺ ion. 2B. In the absence of EECs, ion and water homeostasis is deregulated due to loss of one arm of the PYY-VIP axis. In EEC-deficient small intestine, loss of PYY results in increased cAMP-signaling, increased chloride transport, and increased water and sodium accumulation in the intestinal lumen. Reduced NHE3 transport activity would cause accumulation of cytosolic H⁺ and a decrease in pH. Subsequently, nutrient absorption would be dysregulated, with diminished di-/tri-peptide absorption due to increased intracellular proton accumulation and with increased uptake of glucose due to an exaggerated Na⁺ gradient across the apical membrane. 2A. Na+-coupled glucose transport is deranged in EEC-deficient human and mouse small intestine. Wild type and EEC-deficient human and mouse intestinal tissues were treated with VIP, then 25 mM D-Glucose was added to the luminal chamber. EEC-deficient intestine had an elevated initial response to glucose (mouse, n=28 wild type, n=9 mutant, ***p=0.0005; HIO, n=6 wild type, n=4 mutant, *p=0.02) that was returned to wild type levels by pre-treatment with 10 nM exogenous PYY (mouse, n=7, *p=0.03 from untreated mutant; HIO, n=3). Inhibition of the PYY receptor in wildtype tissues using the NPY1R antagonist BIB03304 caused an abnormal initial response to glucose that mimicked EEC-deficient tissues (mouse, n=12, ***p=0.0004 from untreated; HIO, n=6, *p=0.02 from untreated). Graphs depict the slope of the curve within the boxed area. Error bars are±s.e.m.; statistics calculated by unpaired, two-tailed Student's t-test. B. The levels and subcellular distribution of glucose transporters SGLT1 and GLUT2 are normal in human intestinal tissue lacking EECs. Scale bars=50 μm. 2C. SGLT1 is functional in EEC-deficient human small intestine. Human small intestinal tissue was isolated and transport of glucose in response to saturating amounts of NaCl were measured using the glucose analog 6-NBD 2G. EEC-deficient human small intestinal cells displayed similar total 6-NBDG uptake in the presence of NaCl to wild type human intestinal cells and wild type mouse jejunum cells, demonstrating functional SGLT1-mediated transport. 2D. The ability of SGLT1 to transport Na⁺ is not altered in EEC-deficient enteroids. Enteroids were stained with the Na⁺ fluorescent indicator NaGreen in the presence or absence of 25 mM glucose. The Na⁺ transport activity of SGLT1 in the presence of glucose is similar in both wild type and EEC-deficient epithelium as measured by fluorescence intensity (MFI). Data represents 4 independent experiments. 2E. Total glucose transport is similar in wild-type and EEC-deficient monolayer cultures. Wild-type and EEC-deficient enteroids were cultured as monolayers on transwell inserts and exposed to 25 mM D-glucose with 1 mM fluorescent glucose analog 2-NBDG on the apical surface. The fluorescence intensity of the basal chamber was quantified after 30 minutes (lower graph). The epithelium was then analyzed for 2-NBDG within CDH1-mRuby2-positive epithelium. Data represents 8 independent experiments.

FIG. 3A-3D. H⁺-coupled dipeptide absorption is impaired in EEC-deficient small intestine. 3A. EEC-deficient human and mouse small intestine did not respond to luminal dipeptide in the Ussing chamber (mouse, n=9 wild type, n=6 mutant, ***p=1e-5; human, n=11 wild type, n=5 mutant, **p=0.001). 10 minutes pre-treatment of EEC-deficient tissue with 10 nM exogenous PYY (mouse, n=6), or of wild-type tissue with 300 nM NPY1R inhibitor BIB03304 (mouse, n=9; human, n=6) did not alter the I_{sc} response to Gly-Sar. Error bars are±s.e.m.; statistics calculated by unpaired, two-tailed Student's t-test. 3B. Expression and localization of peptide transporter PEPT1 is unchanged in EEC-deficient human small intestine. Scale bars=50 μm. 3C. The VIP-PYY axis regulates intracellular pH in human small intestinal cells. Wild-type and EEC deficient enteroids were differentiated in the presence of 10 nM VIP for 5-7 days. EEC-deficient enteroids treated with VIP developed an H⁺ imbalance with an acidic cytoplasm whereas concurrent treatment with 10 nM PYY normalized the pH in EEC-deficient enteroids. n=4 independent experiments; ***p=4e-6. Error bars are±s.e.m.; statistics calculated by unpaired, two-tailed Student's t-test. 3D Small intestinal EECs regulate proton transport in a paracrine fashion. Using animals with mosaic loss of EECs Applicant found that regions of epithelium containing ChgA+EECs (arrow) had normal pH and H⁺ transport. Adjacent regions lacking EECs had impaired elevated cytosolic H⁺ as measured by flow cytometry using the fluorescent pH indicator dye pHrodo. There was no difference in pHrodo MFI between mosaic regions in wild-type jejunum (n=8), but a significant increase in pHrodo MFI, indicating relative acidic pH, in EEC-deficient jejunum compared to non-recombined epithelial cells within the same segment of jejunum (n=4, *p=0.04). Error bars are±s.e.m.; statistics calculated by unpaired, two-tailed Student's t-test.

FIG. 4A-4G. Exogenous PYY rescues EEC-deficient mice from malabsorptive diarrhea and restores normal glucose and dipeptide transport. 4A. PYY treatment promotes survival of EEC-deficient mice. Survival curve of wild type (n=100), (n=32) and EEC-deficient mice treated once daily with 10 ug PYY (n=25) beginning at P10. Mice were weaned at P21. 4B. Daily treatment of EEC-deficient mice with PYY reverses intractable diarrhea. As compared to control, EEC-deficient mice have intractable watery diarrhea from birth (given score of 3, gray bar; n=32; ***p=1e-132). Within 48 hours of PYY treatment, EEC-deficient animals had an average score of 1 with slightly soft yet well-defined fecal pellets (n=25, ***p=6e-26 from untreated mutant). Wild-type littermates produce well-defined fecal pellets (given score of 0, black bar; n=100). Error bars are+s.e.m.; statistics calculated by unpaired, two-tailed Student's t-test. 4C. PYY treatment of EEC-deficient animals restored a normal resting Isc to small intestine. Jejunum from wild-type (black), VillinCre; Neurog3flox/flox (gray) and VillinCre; Neurog3flox/flox+PYY injected (red) mice were mounted in the Ussing chamber. Mutant jejunum exhibited a significantly increased basal Isc compared to wild type, which was significantly decreased after in vivo injections of PYY (n=6, ***p=0.0005). Wild type and untreated mutant data points are the same as FIG. 6. Error bars are+s.e.m.; statistics calculated by unpaired, two-tailed Student's t-test. D. Electrogenic response to VIP was elevated in EEC-deficient animals but restored to wild type levels in mutant mice treated with PYY (n=6, ***p=3e-9). Wild type and untreated mutant data points are the same as FIG. 1. Error bars are+s.e.m.; statistics calculated by unpaired, two-tailed Student's t-test. 4E. PYY treatment restores a normal glucose response in EEC-deficient mouse and human intestine. (mouse, n=6, **p=0.002; HIO, n=5, *p=0.04). Wild type and untreated mutant data points are the same as FIG. 2. Error bars are+s.e.m.; statistics calculated by unpaired, two-tailed Student's t-test. 4F. Proton transport is normalized in EEC-deficient animals following PYY treatment. MFI of pHrodo intensity was normalized between EEC-deficient and EEC-rich regions of the mosaic jejunum (n=2). Wild type and untreated mutant data points are the same as FIG. 3. Error bars are+s.e.m.; statistics calculated by unpaired, two-tailed Student's t-test. G. PYY improves dipeptide transport in EEC-deficient mouse and human intestine. Long-term treatment of EEC-deficient animals and animals hosting transplanted HIOs with PYY resulted in improved Isc response to luminal Gly-Sar compared to untreated mutant tissue (mouse, n=6, *p=0.02; HIO, n=5, **p=0.001). Wild type and untreated mutant data points are the same as FIG. 3. Error bars are+s.e.m.; statistics calculated by unpaired, two-tailed Student's t-test.

FIG. 5A-5C. NEUROG3 is required for enteroendocrine cell development in human intestinal organoids. 5A. Human intestinal organoids (HIOs) derived from human pluripotent stem cells with a null mutation in NEUROG3 lacked enteroendocrine cells (EECs) but otherwise had a normal morphology. The epithelial morphology was assessed using a PSC line expressing a CDH1-mRuby2 fusion protein³³ (red, bottom panels) and by co-staining with an anti-CDH1 antibody (red, top panels). Loss of NEUROG3 did not alter markers of intestinal identity (CDX2, purple). Only wild-type (top) and wild-type CDH1-mRuby2 (bottom) HIOs generated Chromogranin A (CHGA)—expressing EECs (green). Scale bars=50 μm. 5B. After maturation in vivo, HIOs develop well-defined crypt-villus architecture. Transplantation of HIOs (˜1 mm) into mice for 10-12 weeks results in growth (1-2 cm), morphogenesis and maturation¹⁶. The epithelium is labeled by CDH1-mRuby2. Scale bar=500 μm. 5C. Transplanted HIOs with disrupted NEUROG3 lacked EECs as marked by CHGA+ but were otherwise morphologically normal. DAPI and CDH1-mRuby2 mark nuclei and epithelium, respectively. Scale bars=100 μm.

FIG. 6A-6B. Ion transport is deranged in EEC-deficient small intestine and can be normalized by PYY. A. There was no significant difference in CFTR or SLC9A3 mRNA expression between enteroids generated from wild-type or EEC-deficient HIOs. n=3. Error bars are+s.e.m. b. PYY modulates basal I_{sc} in human and mouse small intestine. EEC-deficient mouse and human small intestine had significantly higher basal I_{sc} than wild-type (mouse, n=36 wild type, n=11 mutant, *p=0.02; HIO, n=7 wild type, n=12 mutant, *p=0.01) after equilibration in the Ussing chamber. Addition of 10 nM PYY lowered the basal I_{sc} in mutant mouse and human tissue (mouse, n=9, human, n=9) whereas 300 nM NPY1R inhibitor BIB03304 reproducibly increased the basal I_{sc} in wild-type (mouse, n=26, human, n=10). Arrow indicates time of PYY or BIB03304 application to the experiment. One representative trace is shown (mouse). Error bars are±s.e.m.; statistics calculated by unpaired, two-tailed Student's t-test.

FIG. 7. VIP and PYY regulate NHE3 expression. The VIP-PYY axis regulates SLC9A3 expression. After 5-7 days of exposure to VIP, SLC9A3 expression was reduced in wild type (*p=0.04) and in EEC-deficient enteroids (*p=0.02). Exposure to PYY concurrently with VIP in EEC-deficient enteroids restored SLC9A3 expression to not significantly different from untreated. n=4 independent experiments. Error bars are±s.e.m.; statistics calculated by unpaired, two-tailed Student's t-test.

FIG. 8. PYY is abundant in mouse and human small intestine. PYY+EECs (arrows) are abundant in mouse and human small intestine. CDH1 labels epithelium in purple. Scale bars=100 μM.

FIG. 9A-9B. VillinCre; Neurog3^{flox/flox}; Rosa26^{Flox-STOP-flox-dtTomato} mice display incomplete recombination. 9A. Quantification of efficiency of recombination of VillinCre. Jejunum of VillinCre; Neurog3^{flox/flox}; Rosa26^{Flox-STOP-flox-tdTomato} and VillinCre; Neurog3^{+/+}; Rosa26^{Flox-STOP-flox-tdTomato} were subjected to flow cytometry. After doublet discrimination, live, EpCam⁺ cells were analyzed for tdTomato expression. Approximately 6±2.5% of the epithelium did not recombine (n=22). 9B. Representative dot plots and gating strategy from flow cytometric analysis of VillinCre; Neurog3^{+/+}; Rosa26^{Flox-STOP-flox-tdTomato} and VillinCre; Neurog3^{flox/flox}; Rosa26^{Flox-STOP-flox-tdTomato} jejunum.

### DETAILED DESCRIPTION

### Definitions

Unless otherwise noted, terms are to be understood according to conventional usage by those of ordinary skill in the relevant art. In case of conflict, the present document, including definitions, will control. Preferred methods and materials are described below, although methods and materials similar or equivalent to those described herein may be used in practice or testing of the present invention. All publications, patent applications, patents and other references mentioned herein are incorporated by reference in their entirety. The materials, methods, and examples disclosed herein are illustrative only and not intended to be limiting.

As used herein and in the appended claims, the singular forms “a,” “and,” and “the” include plural referents unless the context clearly dictates otherwise. Thus, for example, reference to “a method” includes a plurality of such methods and reference to “a dose” includes reference to one or more doses and equivalents thereof known to those skilled in the art, and so forth.

The term “about” or “approximately” means within an acceptable error range for the particular value as determined by one of ordinary skill in the art, which will depend in part on how the value is measured or determined, e.g., the limitations of the measurement system. For example, “about” may mean within 1 or more than 1 standard deviation, per the practice in the art. Alternatively, “about” may mean a range of up to 20%, or up to 10%, or up to 5%, or up to 1% of a given value. Alternatively, particularly with respect to biological systems or processes, the term may mean within an order of magnitude, preferably within 5-fold, and more preferably within 2-fold, of a value. Where particular values are described in the application and claims, unless otherwise stated the term “about” meaning within an acceptable error range for the particular value should be assumed.

As used herein, the term “effective amount” means the amount of one or more active components that is sufficient to show a desired effect. This includes both therapeutic and prophylactic effects. When applied to an individual active ingredient, administered alone, the term refers to that ingredient alone. When applied to a combination, the term refers to combined amounts of the active ingredients that result in the therapeutic effect, whether administered in combination, serially or simultaneously.

The terms “individual,” “host,” “subject,” and “patient” are used interchangeably to refer to an animal that is the object of treatment, observation and/or experiment. Generally, the term refers to a human patient, but the methods and compositions may be equally applicable to non-human subjects such as other mammals. In some embodiments, the terms refer to humans. In further embodiments, the terms may refer to children.

“Sequence identity” as used herein indicates a nucleic acid sequence that has the same nucleic acid sequence as a reference sequence, or has a specified percentage of nucleotides that are the same at the corresponding location within a reference sequence when the two sequences are optimally aligned. For example a nucleic acid sequence may have at least 50%, 60%, 70%, 75%, 80%, 85%, 90%, 95%, 96%, 97%, 98%, 99%, or 100% identity to the reference nucleic acid sequence. The length of comparison sequences will generally be at least 5 contiguous nucleotides, preferably at least 10, 11, 12, 13, 14, 15, 16, 17, 18, 19, 20, 21, 22, 23, 24, or 25 contiguous nucleotides, and most preferably the full length nucleotide sequence. Sequence identity may be measured using sequence analysis software on the default setting (e.g., Sequence Analysis Software Package of the Genetics Computer Group, University of Wisconsin Biotechnology Center, 1710 University Avenue, Madison, Wis. 53705). Such software may match similar sequences by assigning degrees of homology to various substitutions, deletions, and other modifications.

The ability to absorb ingested nutrients is an essential function of all metazoans and utilizes a wide array of nutrient transporters found on the absorptive enterocytes of the small intestine. A unique population of patients has been identified with severe congenital malabsorptive diarrhea upon ingestion of any enteral nutrition¹. The intestines of these patients are macroscopically normal, but lack enteroendocrine cells, a rare population of cells that release bioactive peptides in response to nutrient cues². The mechanism by which enteroendocrine cells integrate nutrient sensing with nutrient absorption by neighboring cells is poorly understood. By using enteroendocrine-deficient human pluripotent stem cell-derived intestinal organoids and mouse models, Applicant has found that vasoactive intestinal peptide and peptide YY, two well-known regulators of ion and water secretion in the colon³, cooperate to regulate ion-coupled absorption of glucose and dipeptides in mouse and human small intestine. Applicant found that administration of peptide YY to enteroendocrine-deficient mice⁴ restored normal electrophysiology, improved glucose and peptide absorption, diminished diarrhea and rescued postnatal survival, suggesting that peptide YY may be used to treat patients with malabsorption. Applicant uncovered a novel role for crosstalk between enteroendocrine cells and the enteric nervous system in integrating nutrient sensing with nutrient absorption in mouse and human small intestine. As EECs are frequently dysregulated in inflammatory bowel and metabolic diseases, the mechanisms by which they modulate nutrient absorption has wide implications.

In one aspect, disclosed herein is a method of treating a malabsorptive disorder. The malabsorptive disorder may be characterized by, in certain aspects, the malabsorption of macronutrients in the intestine. The method may comprise administering peptide YY (PYY) to an individual in need thereof.

Peptide YY, or “PYY” is known and described in the art. The term PYY includes variants of PYY, including variants of the specific sequences disclosed herein. Variants to PYY will be readily determinable using routine methods in the art. For example, it will be appreciated that one or more amino acids may be modified to arrive at a PYY having similar or sufficient activity, that such activity may be readily determined using routine methods, and that the variant may be used with the disclosed methods. In one aspect, the PYY peptide may be PYY(1-36), available from Phoenix Pharmaceuticals: http://www.phoenixpeptide.com/products/view/Peptides/059-03. In one aspect, the PYY peptide may be identical in sequence to that of human PYY, having the following sequence: Tyr-Pro-Ile-Lys-Pro-Glu-Ala-Pro-Gly-Glu-Asp-Ala-Ser-Pro-Glu-Glu-Leu-Asn-Arg-Tyr-Tyr-Ala-Ser-Leu-Arg-His-Tyr-Leu-Asn-Leu-Val-Thr-Arg-Gln-Arg-Tyr-NH2 (SEQ ID NO 7) as described in https://www.sciencedirect.com/science/article/pii/S000691X88803085. In one aspect, the PYY peptide may Tyr-Pro-Ala-Lys-Pro-Glu-Ala-Pro-Gly-Glu-Asp-Ala-Ser-Pro-Glu-Glu-Leu-Ser-Arg-Tyr-Tyr-Ala-Ser-Leu-Arg-His-Tyr-Leu-Asn-Leu-Val-Thr-Arg-Gln-Arg-Tyr-NH2 (SEQ ID NO 8), or a variant thereof.

In one aspect, the administration of PYY improves absorption of nutrients in the small intestine. In one aspect, the administration may improve absorption of one or both of amino acids and carbohydrates in the intestines, particularly the small intestine. In one aspect, the administration may improve glucose absorption in the intestines, particularly the small intestine.

The methods may be used to treat a variety of disease states or conditions. For example, the method may be used to treat a malabsorptive disorder, for example, one or more of enteric anendocrinosis, short gut syndrome, enteric pathogen infection, malnutrition, genetic causes of malabsorption, Celiac disease, malabsorptive diarrhea, inflammatory bowel diseases such as Chron's and colitis, or any combination thereof. In one aspect, the individual to be treated may be one who is Enteroendocrine cells (EEC)—deficient, or who otherwise has a decreased EEC population, or decreased function of EECs. In one aspect, the individual treated using the disclosed methods may be an individual who is dependent on parenteral nutrition.

In certain aspects, the disclosed methods may include administration until carbohydrate and/or amino acid absorption in the intestine, in particular the small intestine, is improved or normalized. In other aspects, the administration may be carried out until dipeptide absorption in the intestines, in particular the small intestine, is improved or normalized.

In one aspect, the administration of PYY or a variant thereof may be in an amount of from about 1 mg/kg to about 200 mg/kg, or from about 5 mg/kg up to about 100 mg/kg, or from about 10 mg/kg to about 50 mg/kg. In certain aspects, the administration may be in an amount of up to or including about 200 pmol/kg lean body mass.

In certain aspects, one or more additional actives may be administered with PYY. For example, in one aspect, a dipeptidyl peptidase-4 (“DPP4”) inhibitor may be administered before, after, or concurrently with administration of PYY. Dipeptidyl peptidase-4 (or IV) cleaves the first two residues (Tyr-Pro) from the full-length PYY(1-36), converting to (3-36). (3-36) has anorectic effects on the central nervous system which is desirable to avoid, and (1-36) is more potent in the gut epithelium. DPP4 has many other peptide targets, and DPP4 inhibition is in clinical use for one of its other targets, GLP-1, for the treatment of type 2 diabetes. See, e.g., http://www.emdmillipore.com/US/en/product/DPP-IV-Inhibitor,MM_NF-DPP4-010. Dosages may include from about 1 to about 500 mg, or from about 2.5 mg to about 100 mg, though it is to be understood that desirable doses may be determined by routine experimentation and may be unique to the individual. The DPP4 inhibitor may be administered in an amount sufficient to prevent or reduce PYY cleavage. DPP4 inhibitors are known in the art and may include one or more of sitagliptin, vildagliptin, saxagliptin, alogliptin, linagliptin, and combinations thereof. In a further aspect, the additional active may be a vasoactive intestinal peptide (VIP) inhibitor. In one aspect, the VIP inhibitor may be VIP(6-28) and [D-p-Cl-Phe⁶,Leu¹⁷]-VIP. Suitable doses of a VIP inhibitor may be from about 1 mg/kg to about 200 mg/kg, or from about 5 mg/kg up to about 100 mg/kg, or from about 10 mg/kg to about 50 mg/kg, preferably up to or including about 200 pmol/kg lean body mass.

In one aspect, the PYY may be administered via a dosing regime, wherein one or more doses are administered to an individual in need thereof over a period of time. PYY may be administered for at least one day, or at least two days, or at least three days, or at least four days, or at least five days, or at least six days, or at least seven days, or from about one day to about 30 days, or for at least two months, or at least three months, or at least four months, or at least five months, or at least six months, or until improvement or resolution of malabsorption of a nutrient selected from one or both of carbohydrates and amino acids.

In a further aspect, a medicament for improving small and/or large intestine absorption of a macronutrient selected from one or both of a nutrient and a carbohydrate, or for the treatment of obesity is disclosed, wherein said medicament may comprise peptide YY (PYY). In certain aspects, the medicament may further comprise a Dipeptidyl peptidase-4 (“DPP4”) inhibitor. The DPP4 inhibitor may be selected from one or more of sitagliptin, vildagliptin, saxagliptin, alogliptin and linagliptin. In a further aspect, the medicament may comprise a vasoactive intestinal peptide (VIP) inhibitor. In one aspect, the VIP inhibitor may be VIP(6-28) and [D-p-Cl-Phe⁶,Leu¹⁷]-VIP.

In a yet further aspect, disclosed herein is a composition comprising parenteral nutrition and peptide YY (PYY). The PPY may be present in the parenteral nutrition an amount sufficient to improve absorption of one or both of amino acids and carbohydrates in the intestine, more particularly the small intestine, more particularly in an amount that improves/enhances absorption of glucose in the small intestine. The composition may further comprise a vasoactive intestinal peptide (VIP) inhibitor. In one aspect, the VIP inhibitor may be VIP(6-28) and [D-p-Cl-Phe⁶,Leu¹⁷]-VIP.

In a yet further aspect, disclosed herein is a method for treating obesity. The method may comprise administering a peptide YY (PYY) inhibitor to an individual in need thereof. In one aspect, the PYY inhibitor may be BIB03304. NPY1R is the PYY receptor in the gut, which is inhibited by BIBO3304, having the following structure: N-[(1R)-1-[[[[4-[[(Amirtocarbortyl)amino]methyl]phenyl]methyl]amino]carbonyl]-4-[(aminoiminomethyl)amino]butyl]-α-phenyl-benzeneacetamide ditrifluoroacetate (C₂₉H₃₅N₇O₃.2CF₃CO₂H). The method may further comprise administration of a vasoactive intestinal peptide (VIP) inhibitor, such as VIP(6-28) and [D-p-Cl-Phe⁶,Leu¹⁷]-VIP. In a further aspect, the method may further comprise administration of a Dipeptidyl peptidase-4 (“DPP4”) inhibitor, for example, one or more of sitagliptin, vildagliptin, saxagliptin, alogliptin and linagliptin.

### Pharmaceutical Compositions

In one aspect, active agents provided herein may be administered in an dosage form selected from intravenous or subcutaneous unit dosage form, oral, parenteral, intravenous, and subcutaneous. In some embodiments, active agents provided herein may be formulated into liquid preparations for, e.g., oral administration. Suitable forms include suspensions, syrups, elixirs, and the like. In some embodiments, unit dosage forms for oral administration include tablets and capsules. Unit dosage forms configured for administration once a day; however, in certain embodiments it may be desirable to configure the unit dosage form for administration twice a day, or more.

In one aspect, pharmaceutical compositions are isotonic with the blood or other body fluid of the recipient. The isotonicity of the compositions may be attained using sodium tartrate, propylene glycol or other inorganic or organic solutes. An example includes sodium chloride. Buffering agents may be employed, such as acetic acid and salts, citric acid and salts, boric acid and salts, and phosphoric acid and salts. Parenteral vehicles include sodium chloride solution, Ringer's dextrose, dextrose and sodium chloride, lactated Ringer's or fixed oils. Intravenous vehicles include fluid and nutrient replenishers, electrolyte replenishers (such as those based on Ringer's dextrose), and the like.

Viscosity of the pharmaceutical compositions may be maintained at the selected level using a pharmaceutically acceptable thickening agent. Methylcellulose is useful because it is readily and economically available and is easy to work with. Other suitable thickening agents include, for example, xanthan gum, carboxymethyl cellulose, hydroxypropyl cellulose, carbomer, and the like. In some embodiments, the concentration of the thickener will depend upon the thickening agent selected. An amount may be used that will achieve the selected viscosity. Viscous compositions are normally prepared from solutions by the addition of such thickening agents.

A pharmaceutically acceptable preservative may be employed to increase the shelf life of the pharmaceutical compositions. Benzyl alcohol may be suitable, although a variety of preservatives including, for example, parabens, thimerosal, chlorobutanol, or benzalkonium chloride may also be employed. A suitable concentration of the preservative is typically from about 0.02% to about 2% based on the total weight of the composition, although larger or smaller amounts may be desirable depending upon the agent selected. Reducing agents, as described above, may be advantageously used to maintain good shelf life of the formulation.

In one aspect, active agents provided herein may be in admixture with a suitable carrier, diluent, or excipient such as sterile water, physiological saline, glucose, or the like, and may contain auxiliary substances such as wetting or emulsifying agents, pH buffering agents, gelling or viscosity enhancing additives, preservatives, flavoring agents, colors, and the like, depending upon the route of administration and the preparation desired. See, e.g., “Remington: The Science and Practice of Pharmacy”, Lippincott Williams & Wilkins; 20th edition (Jun. 1, 2003) and “Remington's Pharmaceutical Sciences,” Mack Pub. Co.; 18th and 19th editions (December 1985, and June 1990, respectively). Such preparations may include complexing agents, metal ions, polymeric compounds such as polyacetic acid, polyglycolic acid, hydrogels, dextran, and the like, liposomes, microemulsions, micelles, unilamellar or multilamellar vesicles, erythrocyte ghosts or spheroblasts. Suitable lipids for liposomal formulation include, without limitation, monoglycerides, diglycerides, sulfatides, lysolecithin, phospholipids, saponin, bile acids, and the like. The presence of such additional components may influence the physical state, solubility, stability, rate of in vivo release, and rate of in vivo clearance, and are thus chosen according to the intended application, such that the characteristics of the carrier are tailored to the selected route of administration.

For oral administration, the pharmaceutical compositions may be provided as a tablet, aqueous or oil suspension, dispersible powder or granule, emulsion, hard or soft capsule, syrup or elixir. Compositions intended for oral use may be prepared according to any method known in the art for the manufacture of pharmaceutical compositions and may include one or more of the following agents: sweeteners, flavoring agents, coloring agents and preservatives. Aqueous suspensions may contain the active ingredient in admixture with excipients suitable for the manufacture of aqueous suspensions.

Formulations for oral use may also be provided as hard gelatin capsules, wherein the active ingredient(s) are mixed with an inert solid diluent, such as calcium carbonate, calcium phosphate, or kaolin, or as soft gelatin capsules. In soft capsules, the active agents may be dissolved or suspended in suitable liquids, such as water or an oil medium, such as peanut oil, olive oil, fatty oils, liquid paraffin, or liquid polyethylene glycols. Stabilizers and microspheres formulated for oral administration may also be used. Capsules may include push-fit capsules made of gelatin, as well as soft, sealed capsules made of gelatin and a plasticizer, such as glycerol or sorbitol. The push-fit capsules may contain the active ingredient in admixture with fillers such as lactose, binders such as starches, and/or lubricants, such as talc or magnesium stearate and, optionally, stabilizers.

Tablets may be uncoated or coated by known methods to delay disintegration and absorption in the gastrointestinal tract and thereby provide a sustained action over a longer period of time. For example, a time delay material such as glyceryl monostearate may be used. When administered in solid form, such as tablet form, the solid form typically comprises from about 0.001 wt. % or less to about 50 wt. % or more of active ingredient(s), for example, from about 0.005, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, or 1 wt. % to about 2, 3, 4, 5, 6, 7, 8, 9, 10, 15, 20, 25, 30, 35, 40, or 45 wt. %.

Tablets may contain the active ingredients in admixture with non-toxic pharmaceutically acceptable excipients including inert materials. For example, a tablet may be prepared by compression or molding, optionally, with one or more additional ingredients. Compressed tablets may be prepared by compressing in a suitable machine the active ingredients in a free-flowing form such as powder or granules, optionally mixed with a binder, lubricant, inert diluent, surface active or dispersing agent. Molded tablets may be made by molding, in a suitable machine, a mixture of the powdered active agent moistened with an inert liquid diluent.

In some embodiments, each tablet or capsule contains from about 1 mg or less to about 1,000 mg or more of an active agent provided herein, for example, from about 10, 20, 30, 40, 50, 60, 70, 80, 90, or 100 mg to about 150, 200, 250, 300, 350, 400, 450, 500, 550, 600, 650, 700, 750, 800, or 900 mg. In some embodiments, tablets or capsules are provided in a range of dosages to permit divided dosages to be administered. A dosage appropriate to the patient and the number of doses to be administered daily may thus be conveniently selected. In certain embodiments two or more of the therapeutic agents may be incorporated to be administered into a single tablet or other dosage form (e.g., in a combination therapy); however, in other embodiments the therapeutic agents may be provided in separate dosage forms.

Suitable inert materials include diluents, such as carbohydrates, mannitol, lactose, anhydrous lactose, cellulose, sucrose, modified dextrans, starch, and the like, or inorganic salts such as calcium triphosphate, calcium phosphate, sodium phosphate, calcium carbonate, sodium carbonate, magnesium carbonate, and sodium chloride. Disintegrants or granulating agents may be included in the formulation, for example, starches such as corn starch, alginic acid, sodium starch glycolate, Amberlite, sodium carboxymethylcellulose, ultramylopectin, sodium alginate, gelatin, orange peel, acid carboxymethyl cellulose, natural sponge and bentonite, insoluble cationic exchange resins, powdered gums such as agar, or karaya, or alginic acid or salts thereof.

Binders may be used to form a hard tablet. Binders include materials from natural products such as acacia, starch and gelatin, methyl cellulose, ethyl cellulose, carboxymethyl cellulose, polyvinyl pyrrolidone, hydroxypropylmethyl cellulose, and the like.

Lubricants, such as stearic acid or magnesium or calcium salts thereof, polytetrafluoroethylene, liquid paraffin, vegetable oils and waxes, sodium lauryl sulfate, magnesium lauryl sulfate, polyethylene glycol, starch, talc, pyrogenic silica, hydrated silicoaluminate, and the like, may be included in tablet formulations.

Surfactants may also be employed, for example, anionic detergents such as sodium lauryl sulfate, dioctyl sodium sulfosuccinate and dioctyl sodium sulfonate, cationic such as benzalkonium chloride or benzethonium chloride, or nonionic detergents such as polyoxyethylene hydrogenated castor oil, glycerol monostearate, polysorbates, sucrose fatty acid ester, methyl cellulose, or carboxymethyl cellulose.

Controlled release formulations may be employed wherein the active agent or analog(s) thereof is incorporated into an inert matrix that permits release by either diffusion or leaching mechanisms. Slowly degenerating matrices may also be incorporated into the formulation. Other delivery systems may include timed release, delayed release, or sustained release delivery systems.

Coatings may be used, for example, nonenteric materials such as methyl cellulose, ethyl cellulose, hydroxyethyl cellulose, methylhydroxy-ethyl cellulose, hydroxypropyl cellulose, hydroxypropyl-methyl cellulose, sodium carboxy-methyl cellulose, providone and the polyethylene glycols, or enteric materials such as phthalic acid esters. Dyestuffs or pigments may be added for identification or to characterize different combinations of active agent doses.

When administered orally in liquid form, a liquid carrier such as water, petroleum, oils of animal or plant origin such as peanut oil, mineral oil, soybean oil, or sesame oil, or synthetic oils may be added to the active ingredient(s). Physiological saline solution, dextrose, or other saccharide solution, or glycols such as ethylene glycol, propylene glycol, or polyethylene glycol are also suitable liquid carriers. The pharmaceutical compositions may also be in the form of oil-in-water emulsions. The oily phase may be a vegetable oil, such as olive or arachis oil, a mineral oil such as liquid paraffin, or a mixture thereof. Suitable emulsifying agents include naturally-occurring gums such as gum acacia and gum tragamayth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. The emulsions may also contain sweetening and flavoring agents.

Pulmonary delivery of the active agent may also be employed. The active agent may be delivered to the lungs while inhaling and traverses across the lung epithelial lining to the blood stream. A wide range of mechanical devices designed for pulmonary delivery of therapeutic products may be employed, including but not limited to nebulizers, metered dose inhalers, and powder inhalers, all of which are familiar to those skilled in the art. These devices employ formulations suitable for the dispensing of active agent. Typically, each formulation is specific to the type of device employed and may involve the use of an appropriate propellant material, in addition to diluents, adjuvants, and/or carriers useful in therapy.

The active ingredients may be prepared for pulmonary delivery in particulate form with an average particle size of from 0.1 um or less to 10 um or more, for example, from about 0.2, 0.3, 0.4, 0.5, 0.6, 0.7, 0.8, or 0.9 um to about 1.0, 1.5, 2.0, 2.5, 3.0, 3.5, 4.0, 4.5, 5.0, 5.5, 6.0, 6.5, 7.0, 7.5, 8.0, 8.5, 9.0, or 9.5 um. Pharmaceutically acceptable carriers for pulmonary delivery of active agent include carbohydrates such as trehalose, mannitol, xylitol, sucrose, lactose, and sorbitol. Other ingredients for use in formulations may include DPPC, DOPE, DSPC, and DOPC. Natural or synthetic surfactants may be used, including polyethylene glycol and dextrans, such as cyclodextran. Bile salts and other related enhancers, as well as cellulose and cellulose derivatives, and amino acids may also be used. Liposomes, microcapsules, microspheres, inclusion complexes, and other types of carriers may also be employed.

Pharmaceutical formulations suitable for use with a nebulizer, either jet or ultrasonic, typically comprise the active agent dissolved or suspended in water at a concentration of about 0.01 or less to 100 mg or more of active agent per mL of solution, for example, from about 0.1, 1, 2, 3, 4, 5, 6, 7, 8, 9, or 10 mg to about 15, 20, 25, 30, 35, 40, 45, 50, 55, 60, 65, 70, 75, 80, 85, or 90 mg per mL of solution. The formulation may also include a buffer and a simple sugar (e.g., for protein stabilization and regulation of osmotic pressure). The nebulizer formulation may also contain a surfactant, to reduce or prevent surface induced aggregation of the active agent caused by atomization of the solution in forming the aerosol.

Formulations for use with a metered-dose inhaler device generally comprise a finely divided powder containing the active ingredients suspended in a propellant with the aid of a surfactant. The propellant may include conventional propellants, such as chlorofluorocarbons, hydrochlorofluorocarbons, hydrofluorocarbons, and hydrocarbons. Example propellants include trichlorofluoromethane, dichlorodifluoromethane, dichlorotetrafluoroethanol, 1,1,1,2-tetrafluoroethane, and combinations thereof. Suitable surfactants include sorbitan trioleate, soya lecithin, and oleic acid.

Formulations for dispensing from a powder inhaler device typically comprise a finely divided dry powder containing active agent, optionally including a bulking agent, such as lactose, sorbitol, sucrose, mannitol, trehalose, or xylitol in an amount that facilitates dispersal of the powder from the device, typically from about 1 wt. % or less to 99 wt. % or more of the formulation, for example, from about 5, 10, 15, 20, 25, 30, 35, 40, 45, or 50 wt. % to about 55, 60, 65, 70, 75, 80, 85, or 90 wt. % of the formulation.

In some embodiments, an active agent provided herein may be administered by intravenous, parenteral, or other injection, in the form of a pyrogen-free, parenterally acceptable aqueous solution or oleaginous suspension. Suspensions may be formulated according to methods well known in the art using suitable dispersing or wetting agents and suspending agents. The preparation of acceptable aqueous solutions with suitable pH, isotonicity, stability, and the like, is within the skill in the art. In some embodiments, a pharmaceutical composition for injection may include an isotonic vehicle such as 1,3-butanediol, water, isotonic sodium chloride solution, Ringer's solution, dextrose solution, dextrose and sodium chloride solution, lactated Ringer's solution, or other vehicles as are known in the art. In addition, sterile fixed oils may be employed conventionally as a solvent or suspending medium. For this purpose, any bland fixed oil may be employed including synthetic mono or diglycerides. In addition, fatty acids such as oleic acid may likewise be used in the formation of injectable preparations. The pharmaceutical compositions may also contain stabilizers, preservatives, buffers, antioxidants, or other additives known to those of skill in the art.

The duration of the injection may be adjusted depending upon various factors, and may comprise a single injection administered over the course of a few seconds or less, to 0.5, 0.1, 0.25, 0.5, 0.75, 1, 2, 3, 4, 5, 6, 7, 8, 9, 10, 11, 12, 13, 14, 15, 16, 17, 18, 19, 20, 21, 22, 23, or 24 hours or more of continuous intravenous administration.

In some embodiments, the active agents provided herein may be provided to an administering physician or other health care professional in the form of a kit. The kit is a package which houses a container which contains the active agent(s) in a suitable pharmaceutical composition, and instructions for administering the pharmaceutical composition to a subject. The kit may optionally also contain one or more additional therapeutic agents currently employed for treating a disease state as described herein. For example, a kit containing one or more compositions comprising active agents provided herein in combination with one or more additional active agents may be provided, or separate pharmaceutical compositions containing an active agent as provided herein and additional therapeutic agents may be provided. The kit may also contain separate doses of a active agent provided herein for serial or sequential administration. The kit may optionally contain one or more diagnostic tools and instructions for use. The kit may contain suitable delivery devices, e.g., syringes, and the like, along with instructions for administering the active agent(s) and any other therapeutic agent. The kit may optionally contain instructions for storage, reconstitution (if applicable), and administration of any or all therapeutic agents included. The kits may include a plurality of containers reflecting the number of administrations to be given to a subject.

### EXAMPLES

The following non-limiting examples are provided to further illustrate embodiments of the invention disclosed herein. It should be appreciated by those of skill in the art that the techniques disclosed in the examples that follow represent approaches that have been found to function well in the practice of the invention, and thus may be considered to constitute examples of modes for its practice. However, those of skill in the art should, in light of the present disclosure, appreciate that many changes may be made in the specific embodiments that are disclosed and still obtain a like or similar result without departing from the spirit and scope of the invention.

Enteroendocrine cells (EECs) are a rare population of cells found in the gastrointestinal epithelium that sense nutrients that are passing through the gut and in response secrete more than 20 distinct biologically active peptides. These peptides act in an endocrine or paracrine fashion to regulate all aspects of nutrient homeostasis including satiety, mechanical and chemical digestion, nutrient absorption, storage and utilization². Humans' and mice⁴ with genetic mutations that impact formation or function of EECs have intractable malabsorptive diarrhea, metabolic acidosis, and require parenteral nutrition or small-bowel transplant for survival. These findings were the first to link EECs to the absorption of macronutrients; however, the mechanism by which EECs contribute to this vital process is unknown. Poor absorption of macronutrients is a global health concern, with underlying etiology including short-gut syndrome, enteric pathogen infection, and malnutrition. Therefore, identification of factors regulating nutrient absorption has significant therapeutic potential.

Absorption of carbohydrate and protein require coordinated activity of nutrient and ion transporters in the small intestine. Glucose is primarily absorbed via sodium-glucose cotransporter SGLT1, which uses a downhill Na⁺ gradient to transport one glucose or galactose molecule with two sodium ions from the lumen into the enterocyte⁵. The majority of dietary protein absorption occurs via PEPT1, which imports di- and tri-peptides coupled with a hydrogen ion⁶. The electrochemical gradients that drive nutrient absorption are maintained in part by ion transporters, including the cystic fibrosis transmembrane receptor (CFTR), which exports chloride⁷, and sodium-hydrogen exchanger NHE3, which maintains intracellular pH⁸. Activity of CFTR and NHE3 are, in turn, regulated by levels of cyclic AMP (cAMP)^{9,10}. Given that most secreted EEC peptides signal via G protein-coupled receptors to effect second messenger cascades, Applicant investigated if EECs coupled nutrient sensing to nutrient absorption by regulating electrogenic transport in neighboring enterocytes. Two well-studied peptides governing ion and water homeostasis in the colon are vasoactive intestinal peptide (VIP) and peptide YY (PYY). VIP, secreted from enteric neurons, signals via the G_{αs}-coupled VIPR1 (VPAC1) on epithelial cells to raise levels of intracellular cAMP. In the colon, EEC-derived PYY acts in a paracrine fashion to lower cAMP via the G_{αi} coupled receptor NPY1R¹¹⁻¹⁴. Applicant posited that the mechanism underlying malabsorptive diarrhea in patients lacking EECs might be due to loss of a similar EEC-ENS regulatory feedback in the small intestine. This would disrupt both the normal ion gradients and ion-coupled nutrient absorption.

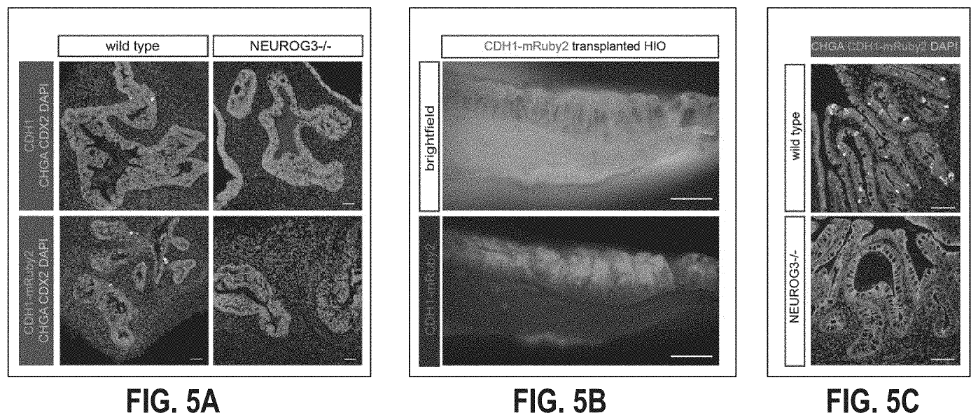

To investigate this, Applicant used EEC-deficient mice⁴ (VillinCre;Neurog3^{flox/flox}) and three different human intestinal tissue models all derived from pluripotent stem cells (PSCs): human intestinal organoids (HIOs) derived in vitro¹⁵, mature intestinal tissue isolated from HIOs that were grown in vivo¹⁶, and epithelial organoids (enteroids) derived from crypts of matured HIO tissues¹⁶. Applicant generated EEC-deficient human intestinal tissue by using PSC lines that had a null mutation in NEUROG3¹⁷, the basic helix-loop-helix transcription factor required for EEC formation in mice¹⁸ and humans¹. As previously reported¹⁹, NEUROG3^{-/-} intestinal tissue completely lacked EECs, but was otherwise normal in appearance (FIG. 5A-5C). EECs and enteric neurons are spatially connected²⁰, can directly synapse^{21,22}, and reciprocally express receptors for gut peptides and neurotransmitters^{23,24}. Furthermore, enterocytes respond to signals from both EECs and the ENS²⁵. To formally test whether the VIP-PYY axis operated in human small intestine, Applicant performed experiments in HIOs without an integrated ENS, wherein Applicant controlled exogenous exposure to a single ENS-derived peptide, VIP.

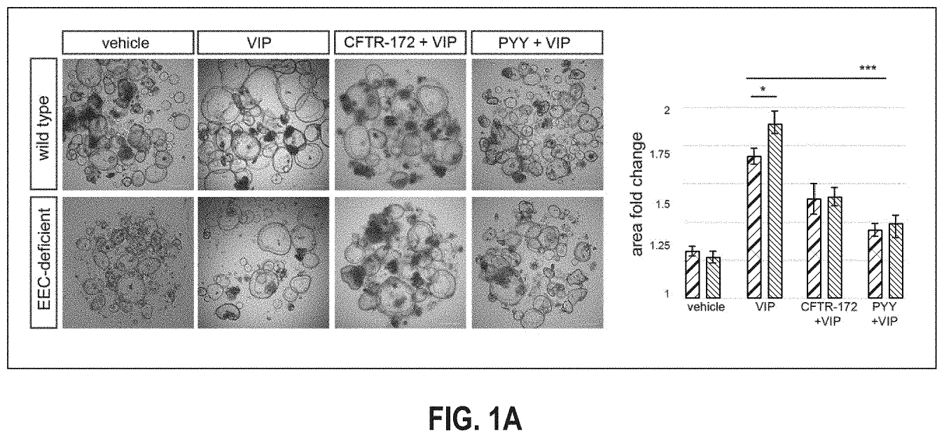

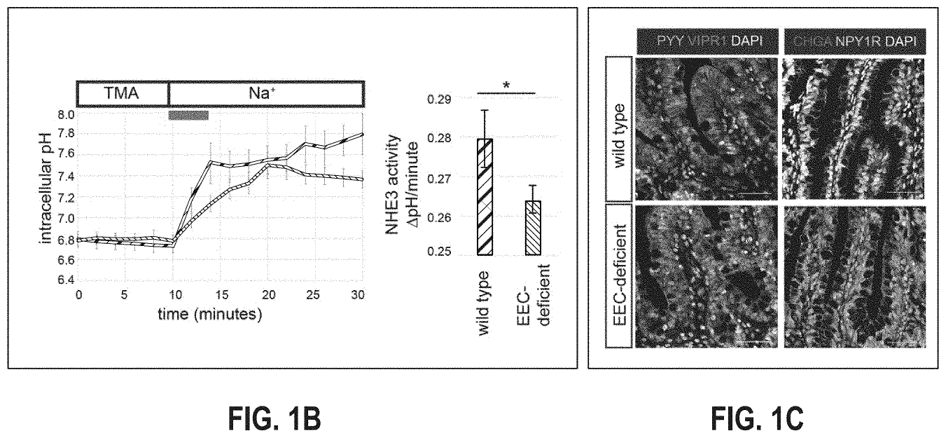

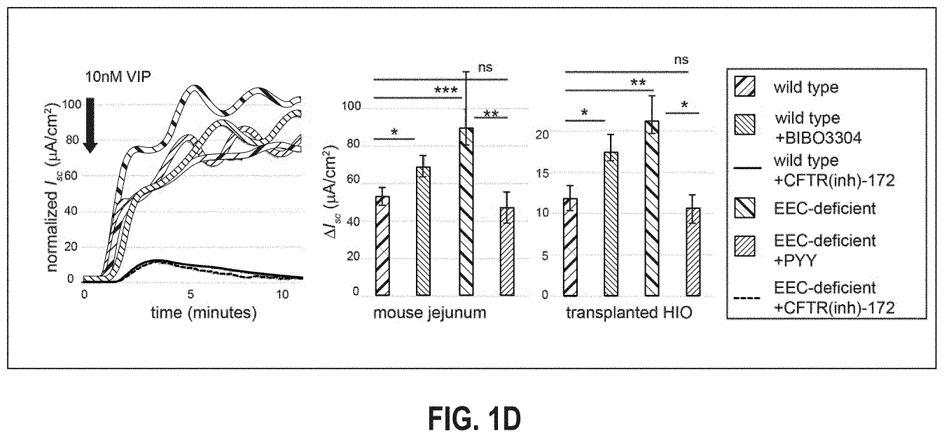

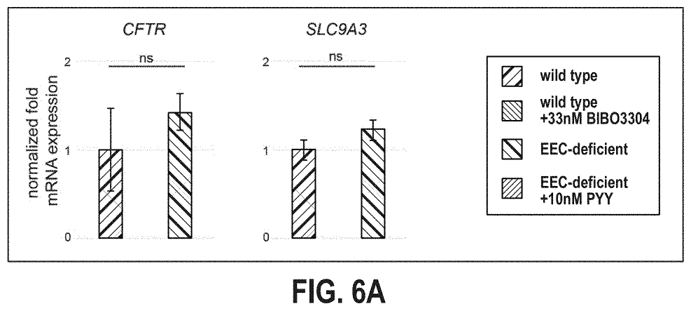

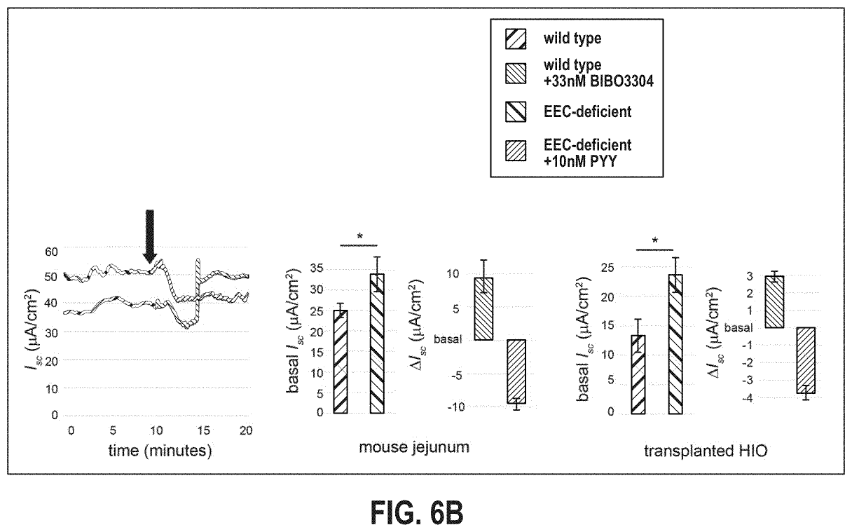

Given that ion and water transport in the colon is regulated by EEC-derived PYY and ENS-derived VIP, Applicant investigated if disruption of the PYY-VIP axis might affect ion and water transport in EEC-deficient small intestine. Applicant measured CFTR-mediated ion and water efflux by quantifying the swelling response²⁶ of enteroids to exogenous VIP. EEC-deficient enteroids swelled significantly more than did wild-type, which was blocked with the CFTR inhibitor CFTR-172 (FIG. 1A). Exogenous PYY also blocked VIP-induced swelling, resulting in a normalized response between wild type and EEC-deficient enteroids (FIG. 1A). Applicant next tested the activity of NHE3 as a measure of Na⁺-dependent intracellular pH recovery after acidic challenge²⁷ and found that EEC-deficient enteroids displayed impaired NHE3 function (FIG. 1B). There was no difference in expression of CFTR, SLC9A3 (encoding NHE3), VIPR1 or NPY1R between wild-type and EEC-deficient small intestine (FIG. 1C and FIG. 6A). Together, these data suggest that EEC-deficient enteroids have an abnormal response to VIP-regulated ion and water transport that can be normalized by the addition of exogenous PYY.

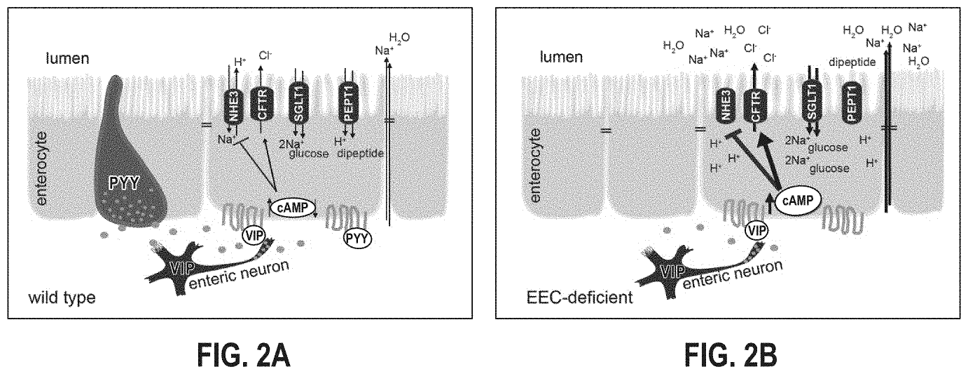

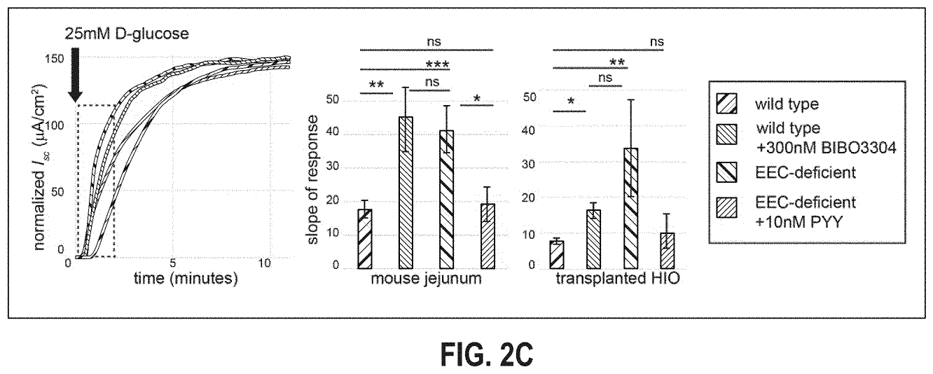

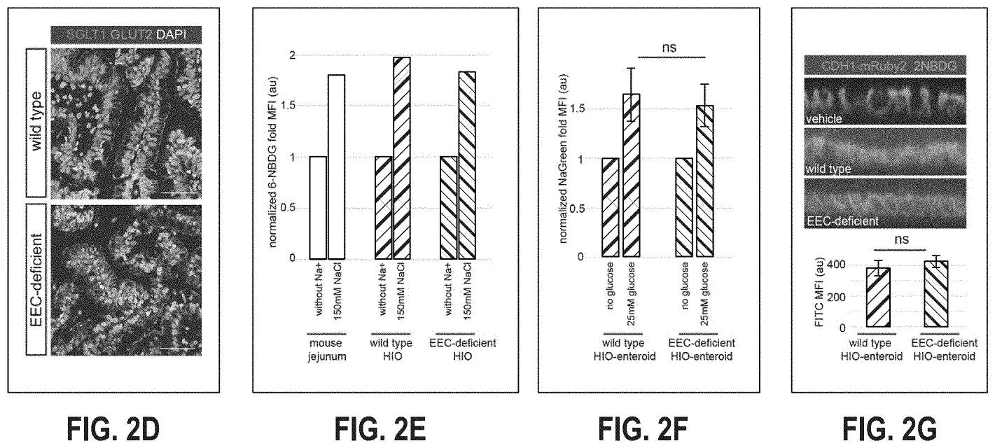

To investigate ion transport activities in full thickness intestinal mucosa, Applicant analyzed matured human intestinal tissue in parallel with jejunum isolated from wild-type and VillinCre;Neurog3^{flox/flox} mice⁴ using a modified Ussing chamber²⁸. Applicant inhibited voltage-gated neuronal firing in mouse jejunum by including tetrodotoxin¹¹ in all experiments so that epithelial response to exogenous VIP could be precisely monitored. Applicant observed that the basal short-circuit current (I_{sc}), a general measure of ion transport, was consistently higher in EEC-deficient mouse and human small intestine compared to normal (FIG. 6B). Addition of exogenous PYY to EEC-deficient jejunum was sufficient to restore the I_{sc} to normal, and chemical inhibition of NPY1R in wild type was sufficient to elevate the I_{sc} (FIG. 6B). Because EEC-deficient enteroids exhibited abnormally elevated swelling in response to exogenous VIP (FIG. 1A), Applicant investigated the electrophysiological response of mouse and human small intestine to VIP in the Ussing chamber. EEC-deficient mouse and human small intestinal tissue displayed an exaggerated I_{sc} response to exogenous VIP compared to wild-type, which was dependent on CFTR (FIG. 1D). Providing PYY to EEC-deficient tissue was sufficient to restore the I_{sc} response to normal, indicating that PYY and VIP coordinately regulate vectorial ion transport in mouse and human small intestine. Blocking endogenous PYY signaling in wild-type tissues resulted in a significantly elevated response to VIP (FIG. 1D), suggesting that endogenous PYY produced in the small intestine was regulating transporter activity in a paracrine manner These data suggest that imbalance of this axis may be a mechanism underlying malabsorptive diarrhea suffered by patients without EECs (FIG. 2A-2B).

While it is known that EECs sense nutrients, the mechanism linking sensing to the control of nutrient absorption is unclear. A hint came from the effects of enteral feeding of EEC-deficient patients, which resulted in a massive diarrheal response. This suggests that an inability to sense luminal nutrients uncoupled the ability to adequately absorb them. To explore this possibility, Applicant evaluated ion-coupled nutrient absorption in EEC-deficient small intestine. Applicant observed an accelerated initial response to luminal glucose in the presence of VIP in EEC-deficient mouse and human intestinal tissues in the Ussing chamber (FIG. 2C), as predicted in the context of deranged electrochemical gradients (FIG. 2A-2B). This recapitulated the exacerbated diarrhea observed in patients without EECs when they were fed with carbohydrate¹. Exogenous PYY restored a normal glucose response in EEC-deficient mouse and human tissue, and inhibition of NPY1R in wild-type caused an exaggerated initial response to glucose (FIG. 2C). These data indicate that PYY is sufficient to modulate glucose absorption in the small intestine. Applicant found no defects in expression of SGLT1 (FIG. 2D) or maximum absorptive competency of Na³⁰-coupled glucose transport (FIG. 2E-2G) in human epithelium without EECs. These data suggest that SGLT1 is competent to absorb glucose, but activity is dysregulated in the context of abnormal ion gradients in the absence of EECs.

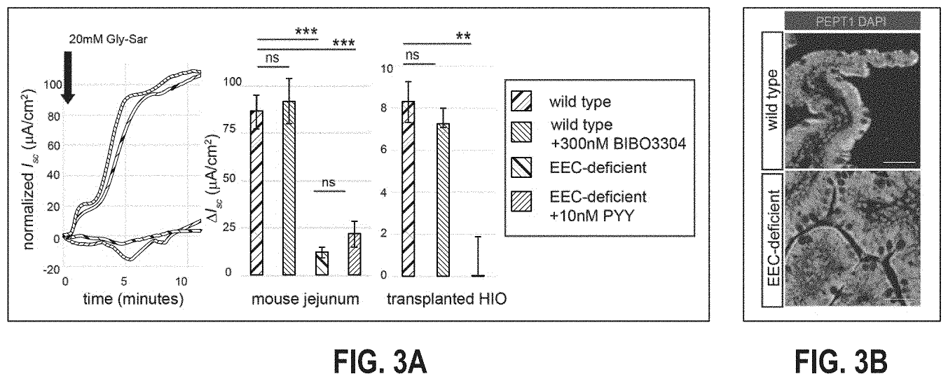

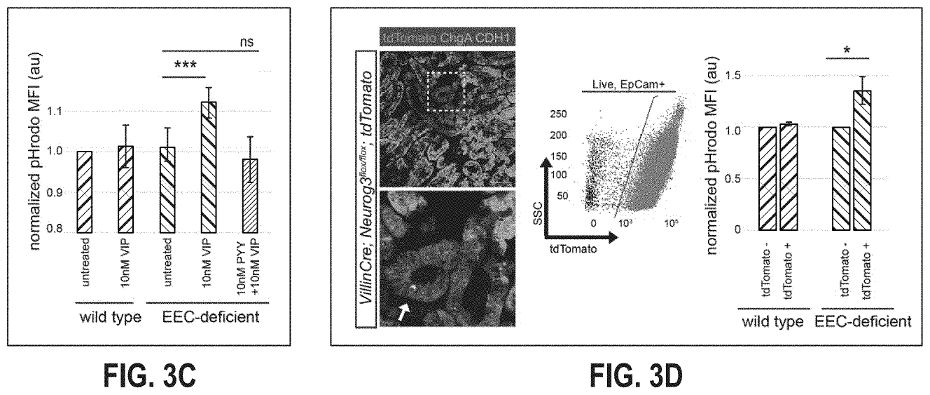

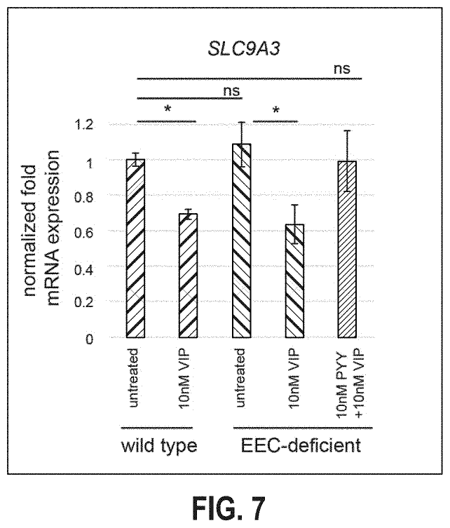

Approximately 80% of ingested amino acids were recovered in the stool of the index EEC-deficient patient¹, suggesting a critical role for EECs in regulating protein absorption. Consistent with this, Applicant observed a striking loss of ion-coupled dipeptide absorption when human and mouse EEC-deficient small intestine were challenged with VIP (FIG. 3A), despite normal expression of PEPT1 (FIG. 3B). VIP has an established role in inhibition of NHE3 and PEPT1-mediated dipeptide absorption^{8,29}, but Applicant was surprised to find that EEC-deficient intestine remained unable to respond to dipeptide when PYY was provided (FIG. 3A). This suggested that dysregulated H⁺ gradients may be a more stable phenotype in EEC-deficient intestine, and not easily reversed by PYY within minutes. To explore this possibility, Applicant treated enteroids with or without PYY for one week in vitro in the presence of VIP. Wild-type enteroids were able to maintain their intracellular pH in the presence of VIP but EEC-deficient enteroids became significantly more acidic (FIG. 3C). However, EEC-deficient enteroids were restored to normal intracellular pH levels and normal SLC9A3 expression (encoding NHE3) in the presence of PYY (FIG. 3C and FIG. 7). This suggested that long-term exposure to an imbalanced EEC-ENS axis dysregulates intestinal physiology, and that, over time, PYY may be sufficient to restore intracellular pH and dipeptide absorption in EEC-deficient small intestine.

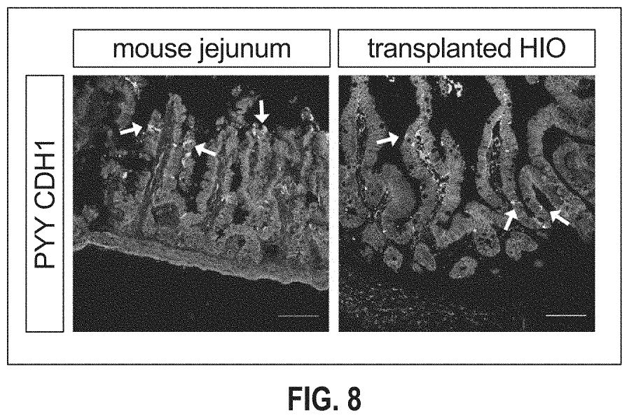

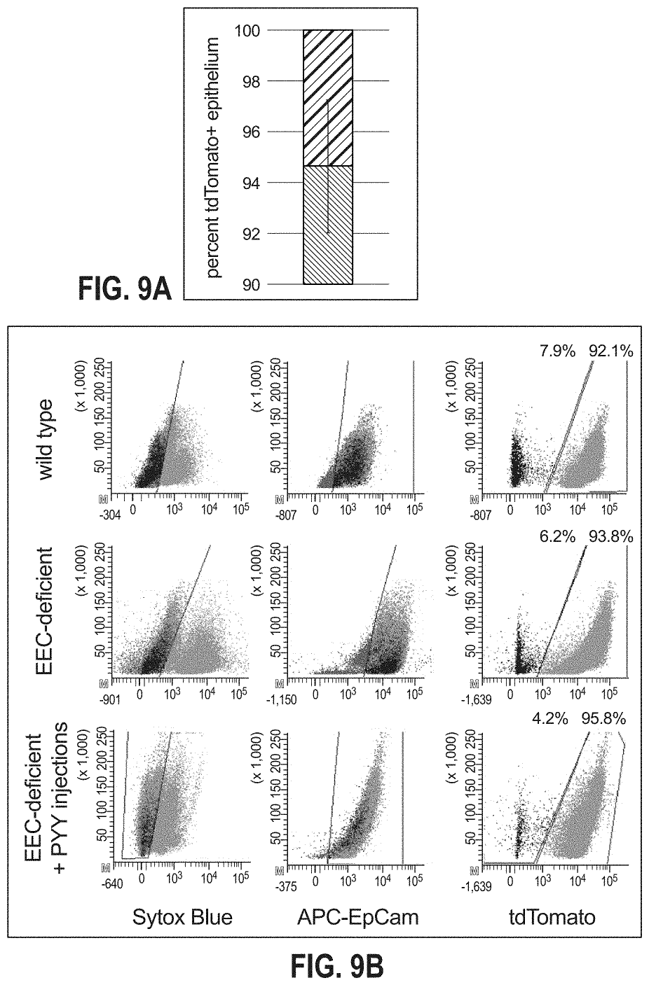

In vivo, the mechanism of action of PYY could be paracrine or endocrine. While PYY-expressing EECs are found in increasing numbers in the distal intestine, they are also abundant in mouse and human small intestine³⁰ (FIG. 8). Moreover, PYY-expressing EECs extend long basal processes which underlie several neighboring epithelial cells^{20,22}, raising the possibility that they may exert paracrine effects on nearby enterocytes. Applicant investigated whether the effects of PYY on ion transport in the small intestine occurred via a paracrine mechanism by exploiting the mosaicism of VillinCre to compare intracellular pH in EEC-deficient and EEC-rich jejunum of the same mouse (FIG. 3C and FIGS. 9A and 9B). EEC-deficient epithelial cells displayed a significantly more acidic intracellular pH than their neighboring EEC-rich epithelial cells (FIG. 3C and FIGS. 9A and 9B), indicating that EECs control local H⁺ transport and dipeptide responsiveness in the small intestine via paracrine, not endocrine, mechanisms.

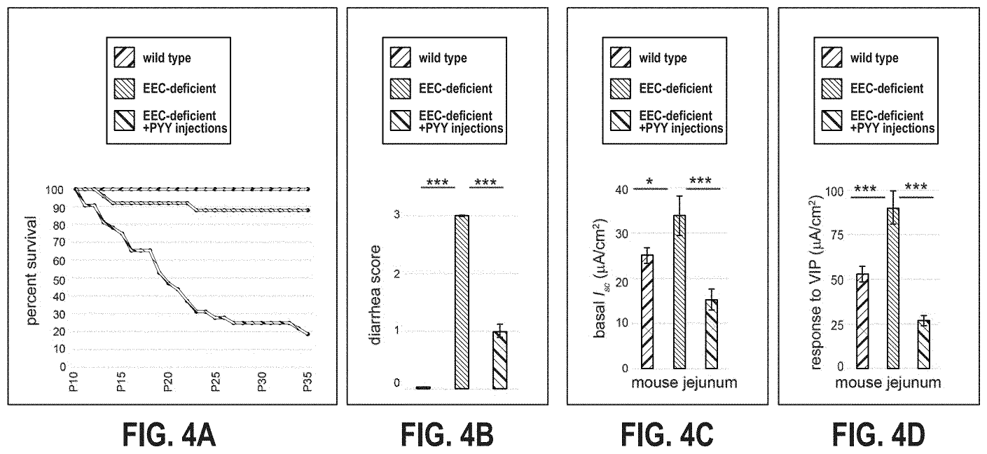

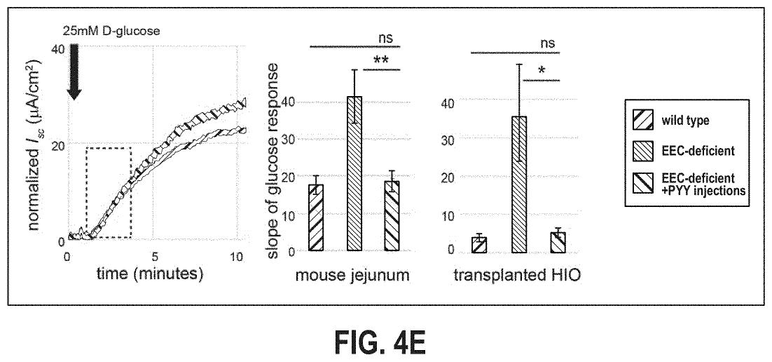

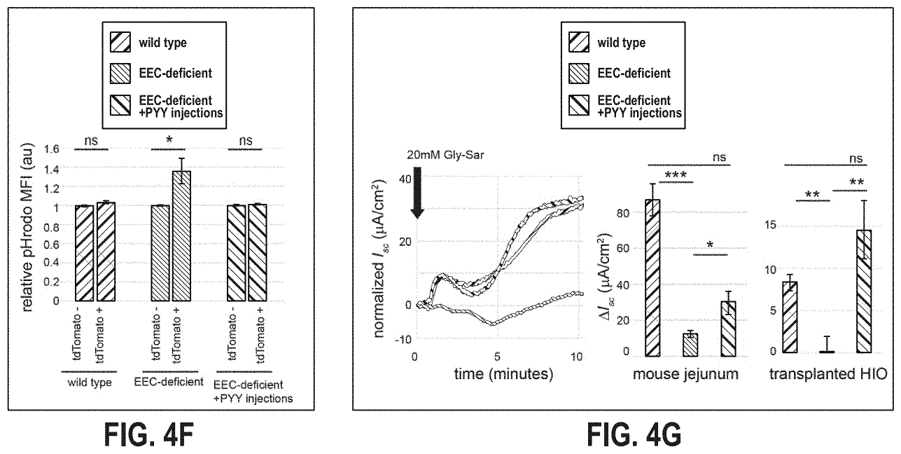

The above data suggested that long-term treatment with PYY may restore normal carbohydrate and amino acid absorption the intestines of EEC-deficient patients. As a preclinical model of EEC-deficiency, Applicant used VillinCre;Neurog3^{flox/flox} mice that suffer from malabsorptive diarrhea, resulting in impaired postnatal survival⁴ (FIG. 4A-4B). Daily treatment of mice began at postnatal day 10 with 10 μg PYY(1-36) by intraperitoneal injection. PYY can be converted to PYY(3-36) by the protease DPP4³¹, and this form of PYY has potent anorexic effects in the brain³². Applicant therefore co-injected PYY(1-36) and a DPP4 inhibitor to prevent PYY cleavage and to better target the epithelial NPY1R receptor that preferentially binds the 1-36 form^{11,13,31}. Patients with EEC-deficiency die without total parenteral nutrition, and similarly very few EEC-deficient mice survive without treatment within the first few weeks. However, PYY injections dramatically improved mutant survival up to 88% (FIG. 4A). Moreover, PYY treatment reduced diarrhea and improved fecal output of mutant mice to either be indistinguishable from wild type or only slightly wet but well-defined pellets (FIG. 4B).

Applicant investigated if the animals that survived in response to PYY injections had restored electrophysiology and improved nutrient absorption in the small intestine. Applicant found that PYY-injections restored the basal I_{sc} of jejunum to normal (FIG. 4C). Additionally, the response to VIP (FIG. 4D) and the response to luminal glucose (FIG. 4E) were both normalized indicating that PYY injections stably restored electrophysiology. Importantly, mice received their last injection of PYY approximately 16 hours prior to sacrifice, demonstrating sustained action of the peptide in vivo. The rescue of EEC-deficient intestinal tissue also extended to the human model, where EEC-deficient HIOs were grown and matured in vivo and then host animals were injected with exogenous PYY for 10 days prior to harvest. These EEC-deficient HIOs exposed to PYY demonstrated electrogenic response to glucose that was indistinguishable from wild-type (FIG. 4E). Lastly, Applicant investigated whether the PYY treated groups had improved amino acid absorption as measured by H⁺ export and response to the dipeptide Gly-Sar. By administering PYY to the mosaic EEC-deficient reporter mice, Applicant found PYY injections restored intracellular pH in EEC-deficient intestinal cells to normal levels which would support PEPT1-mediated dipeptide absorption (FIG. 4F). In support of this, PYY-injected mouse and human small intestine displayed a significantly improved electrogenic response to dipeptides (FIG. 4G), indicating that dipeptide absorption was restored. These data demonstrated functional efficacy of PYY on improved ion and nutrient transport in EEC-deficient intestine.

These data suggest that PYY is sufficient to rescue the electrophysiology and absorptive function of mouse and human small intestine in the absence of all other EEC peptides. Moreover, the improvements in glucose and dipeptide absorption suggest that PYY may be a viable therapeutic option for other malabsorptive disorders, such as short-gut syndrome, malnutrition and enteric pathogen infection. Loss of EECs resulted in profound imbalance of ion transport in the small intestine, with subsequent impairment of nutrient absorption. Moreover, macronutrient absorption in mice and humans is regulated, in part, by an unappreciated PYY-VIP axis in the small intestine that operates in a paracrine fashion. These findings lend some clarity on how EECs integrate their nutrient sensing function with nutrient absorption, providing a new way to approach management of diseases, such as obesity and inflammatory bowel, in which EECs are commonly dysregulated.

### Methods

Pluripotent stem cell culture and directed differentiation of HIOs

Human embryonic stem cell (ESC) line WA01 (H1) was purchased from WiCell. Applicant used H1 cells with a CRISPR/Cas9 generated null mutation in NEUROG3 as previously described¹⁷. Additionally, Applicant inserted the CDH1-mRuby2 reporter construct³³ into NEUROG3−/− H1 hESCs. CDH1-mRuby2 and non-reporter hESCs were used interchangeably. hESCs were maintained in feeder-free culture. Cells were plated on hESC-qualified Matrigel (BD Biosciences, San Jose, Calif.) and maintained at 37° C. with 5% CO₂ with daily removal of differentiated cells and replacement of mTeSR1 media (STEMCELL Technologies, Vancouver, Canada). Cells were passaged routinely every 4 days using Dispase (STEMCELL Technologies). HIOs were generated according to protocols established in our lab^{15,34}.

### In Vivo Transplant of HIOs

28-35 days after spheroid generation, HIOs were removed from Matrigel and transplanted under the kidney capsule of immune deficient NOD.Cg-Prkdc^{scid}I12rg^{tmIWjl}/SzJ (NSG) mice as previously described¹⁶. NSG mice were maintained on Bactrim chow for a minimum of 2 weeks prior to transplantation and thereafter for the duration of the experiment (8-14 weeks).

### Generation and Maintenance of HIO-Derived Enteroids

After approximately 10 weeks of in vivo growth, crypts were isolated from transplanted HIOs and plated in 3D as previous described³⁵. To promote growth, enteroids were maintained in Human IntestiCult components A+B (STEMCELL Technologies). To promote differentiation, HIOEs were cultured in 1:1 IntestiCult component A: Advanced DMEM/F12 with 15 mM HEPES for 5-7 days. Undifferentiated enteroids were passaged every 7-10 days into fresh Matrigel (Corning) using a 25Gx½ needle.

### Immunofluorescence

Tissue was fixed in 4% paraformaldehyde, cryopreserved in 30% sucrose, embedded in OCT, and frozen at −80° C. until cryosectioned. 8 μm cryosections were mounted on Superfrost Plus slides and permeabilized, blocked and stained according to standard protocol. Primary antibodies used are listed in the table below, and all secondary antibodies were conjugated to Alexa Fluor 488, 546/555/568 or 647 (Invitrogen). Images were acquired using a Nikon A1 GaAsP LUNV inverted confocal microscope and NIS Elements software (Nikon).

### Primary Antibodies

| Primary antibody | Company | Host | Dilution |
|---|---|---|---|
| CDX2 | BioGenex | Mouse | 1:300 |
| CDX2 | Cell Marquis | Rabbit | 1:500 |
| Chromogranin A | DSHB | Mouse | 1:500 |
| Chromogranin A | ImmunoStar | Rabbit | 1:250 |
| E-Cadherin (CDH1) | R&D | Goat | 1:500 |
| GLUT2 | Santa Cruz | Goat | 1:500 |
| Muc2 | Santa Cruz | Rabbit | 1:250 |
| NPY1R | Abcam | Rabbit | 1:250 |
| PEPT1 | Santa Cruz | Rabbit | 1:500 |
| PYY | Abcam | Rabbit | 1:1000 |
| SGLT1 | Santa Cruz | Rabbit | 1:250 |
| VIPR1 | Millipore | Mouse | 1:200 |

qPCR

RNA was extracted using Nucleospin RNA extraction kit (Macharey-Nagel) and reverse transcribed into cDNA using Superscript VILO (Invitrogen) according to manufacturer's instruction. qPCR primers were designed using NCBI PrimerBlast. Primer sequences are listed in the table below. qPCR was performed using Quantitect SYBR® Green PCR kit (QIAGEN) and a QuantStudio 3 Flex Real-Time PCR System (Applied Biosystems). Relative expression was determined using the AACt method and normalizing to PPIA (cyclophilin A). Samples from at least three independent passages were used for quantification.

### Primer Sequences

| | |
|---|---|
| PPIA (CPHA) FWD | CCCACCGTGTTCTTCGACATT (SEQ ID NO: 1) |
| PPIA (CPHA) REV | GGACCCGTATGCTTTAGGATGA (SEQ ID NO: 2) |
| CFTR FWD | GGCACCCAGAGTAGTAGGTC (SEQ ID NO: 3) |
| CFTR REV | AGGCGCTGTCTGTATCCTTT (SEQ ID NO: 4) |
| SLC9A3 (NHE3) FWD | GCTGGTCTTCATCTCCGTGT (SEQ ID NO: 5) |
| SLC9A3 (NHE3) REV | CCAGAGGCTTGATGGTCAGG (SEQ ID NO: 6) |

### Swelling Assay

Enteroids were plated in 10 μL Matrigel on an 8-chamber glass bottom slide (Ibidi) and maintained as described above. 3-5 days post-plating, the slide was mounted on an inverted confocal microscope (Nikon) fitted with an incubation chamber set to 37° C. and 5% CO₂. Media was changed to include 10 nM VIP (Tocris). In some experiments, the media was changed 24 hours prior to imaging to include 20 μM CFTR (inh)-172 (Millipore Sigma) or 10 nM PYY (Phoenix Pharmaceuticals). Images were acquired every 5 minutes at 4× magnification. After 6 hours, some HIOEs swelled to the point of bursting; therefore, Applicant used images acquired at time 0 and at 6 hours for quantification. The area of 10 representative enteroids per well was quantified using NIS Elements software at both time points. The outline of individual enteroids was traced manually and the area calculated by NIS Elements. Fold change at 6 hours over baseline was reported. Data include a minimum of three independent experiments per condition.

### NHE3 Activity Assay

NHE3 activity was determined as previously described²⁷ with minor modifications. Enteroids were plated in 5 μL Matrigel on an 8-chamber glass bottom slide (Ibidi) and maintained as described above. 3-5 days post-plating, media was changed to Na⁺ media containing 5 μM SNARF-4F 5-(and-6)-carboxylic acid, acetoxymethyl ester, acetate (Molecular Probes) and allowed to incubate for 30 minutes. The slide was then mounted on an inverted confocal microscope (Nikon), fitted with an incubation chamber set to 37° C. and 5% CO₂. Fresh Na⁺ media was provided before image acquisition Images were acquired every 2 minutes for 2 hours at 10×magnification with excitation at 488 nm and emission at 561 nm and 640 nm. Media was changed to NH₄Cl to acid-load the epithelium, then to tetramethylammonium (TMA) media to withdraw Na⁺. Na⁺ containing media was then added and NHE3 activity quantified as a measure of initial pH recovery. 1 mM probenecid and 5 μM SNARF were present in all buffers, and all buffers were set to pH 7.4. Intracellular pH was calibrated using the Intracellular pH Calibration Buffer kit (Invitrogen) at pH 7.5, 6.5 and 5.5 in the presence of 10 μM valinomycin and 10 μM nigericin at the conclusion of each experiment. The ratio of 561/640 was determined using NIS Elements software by drawing a region of interest and quantifying the fluorescence intensity of each wavelength over the period of the experiment. A minimum of 3 enteroids in 3 wells over two independent passages were quantified. The ratio of 561/640 was converted to intracellular pH using the equation provided by the manufacturer.

Na⁺ media: 130 mM NaCl, 5 mM KCl, 2 mM CaCl₂, 1 mM MgSO₄, 20 mM HEPES, 5 mM NaOH, 1 mM (Na)PO₄, 25 mM D-glucose

NH₄Cl media: 25 mM NH₄Cl, 105 mM NaCl, 2 mM CaCl₂, 1 mM MgSO₄, 20 mM HEPES, 8 mM NaOH, 5 mM KCl, 1 mM (Na)PO₄, 25 mM D-glucose

TMA media: 130 mM TMA-Cl, 5 mM KCl, 2 mM CaCl₂, 1 mM MgSO₄, 20 mM HEPES, 8 mM TMA-OH, 1 mM (TMA)PO₄, 25 mM D-glucose

### Electrophysiology

Electrophysiological experiments were conducted as described²⁸ with minor modifications. Mouse jejunum and transplanted HIOs were dissected and immediately placed in ice-cold Krebs-Ringer solution. Tissues were opened to create a flat epithelial surface. Because seromuscular stripping is associated with release of cyclooxygenases and prostaglandins²⁸, and prostaglandins can stimulate L-cells to release GLP1, GLP2 and PYY³⁶, Applicant performed the Ussing chamber experiments in intestinal tissue with an intact muscular layer. Tissues were mounted into sliders (0.031 cm² area slider, P2307, Physiological Instruments) and placed in an Ussing chamber with reservoirs containing 5 mL buffer (115 mM NaCl, 1.2 mM CaCl₂, 1.2 mM MgCl₂, 25 mM NaHCO₃, 2.4 mM K₂HPO₄ and 0.4 mM KH₂PO₄). The mucosal and serosal tissue surfaces were bathed in the same solution, with the exception of 10 mM glucose in the serosal buffer and 10 mM mannitol in the luminal buffer. Mucosal and serosal reservoir solutions were gassed with 95% O₂ and 5% CO₂ to pH 7.4 and maintained at 37° C. by a circulating water bath behind the reservoir chambers. Electrophysiology parameters were recorded as previously described³⁷. Tissue was allowed to equilibrate to a basal steady-state for a minimum of 30 minutes before the addition of chemicals or peptides. 10 nM tetrodotoxin (Tocris) was added to the serosal buffer bathing mouse intestine to inhibit voltage-gated neuronal firing and allowed to incubate for a minimum of 10 minutes before basal I_{sc} recording. D-glucose and Gly-Sar were added to the luminal side of the chamber once the VIP-induced I_{sc} had stabilized at a maximum value.

TABLE

| | | |
|---|---|---|
| Tetrodotoxin | Tocris | 10 nM final |
| BIBO3304 trifluoroacetate | Tocris | 300 nM final |
| VIP | Tocris | 10 nM final |
| PYY(1-36) | Phoenix Pharmaceuticals | 10 nM final |
| CFTR-172 | Millipore Sigma | 20 μM |
| D-glucose | Sigma Aldrich | 25 mM final |
| Gly-Sar | Sigma Aldrich | 20 mM final |

### Glucose Uptake Assays

### 6-NBDG

Transplanted HIOs were removed from the murine kidney, bisected to expose the lumen, and incubated with 100 mM 6-(N-(7-Nitrobenz-2-oxa-1,3-diazol-4-yl)Amino)-2-Deoxyglucose (6-NBDG) (Life Technologies) in 10 nM Tris/HEPES buffer containing 150 mM KCl or 150 mM NaCl for 30 minutes at 37° C. Tissues were washed with ice-cold 10 mM Tris/HEPES buffer, then dissociated to single-cell suspension in 5 mL Tryple Select (Gibco)+10 μM Y-27632 (Tocris), filtered, and subjected to analysis by flow cytometry.

### Sodium Green

HIOEs were differentiated for 5-7 days, then were removed from Matrigel and enzymatically dissociated into single-cell suspension using 0.25% Trypsin-EDTA. Each cell preparation was split into two samples: one incubated with 25 mM D-glucose and one incubated in the absence of glucose. Each sample was incubated in Live Cell Imaging Solution (Invitrogen) containing 5 μM final concentration of Sodium Green tetraacetate (Molecular Probes) for 30 minutes at 37° C., washed with ice-cold PBS and analyzed by flow cytometry.

2-NBDG on Transwell filters

Undifferentiated enteroids that were “ready to split” were dissociated and plated on transwell inserts (Corning) as previously described³⁸, with the exception of first coating the transwells with Collagen IV (Sigma-Aldrich). 300,000 cells were plated per 6.5 mm transwell insert. Differentiation was initiated at 24 hours post-plating and monolayers were analyzed after 5-7 days. 1 mM fluorescent glucose analog 2-(N-(7-Nitrobenz-2-oxa-1,3-diazol-4-yl)Amino)-2-Deoxyglucose (2-NBDG, Life Technologies) was diluted in Live Cell Imaging Solution (Invitrogen) containing 25 mM D-glucose, added to the apical surface of HIOE monolayers and the fluorescence intensity of fresh Live Cell Imaging Solution in the basal chamber was quantified after 30 minutes at 37° C. Intact barrier function was confirmed by co-incubation, quantification and exclusion of Cascade Blue conjugated 3000 MW dextran (Life Technologies) in every experiment.

### Intracellular pH Assay

Enteroids were differentiated for 5-7 days in the presence of vehicle (water), 10 nM VIP (Tocris) or 10 nM PYY (Phoenix Pharmaceuticals) and 10 nM VIP. On the final day, enteroids were removed from Matrigel and enzymatically dissociated into single-cell suspension using 0.25% Trypsin-EDTA. Cell suspensions were counted and equal cell numbers of dissociated HIOEs were incubated in pHrodo Green AM Intracellular pH indicator (ThermoFisher Scientific) according to manufacturer's directions for 30 minutes at 37 C., washed with 1X PBS, and analyzed by flow cytometry.

### Flow Cytometry

After mechanical and enzymatic dissociation, tissues were filtered through a 40 μm cell strainer to obtain a single-cell suspension. In all experiments, samples were labeled with either CDH1-mRuby2 or Anti-EpCam-APC (BD Biosciences) to distinguish epithelial cells and incubated with SYTOX Blue dead cell stain (Life Technologies) or 7-AAD (BD Pharmingen). Forward scatter and side scatter were used to discriminate doublets and cellular debris. A minimum of 50,000 events per sample was recorded using an LSR Fortessa flow cytometer (BD Biosciences) and data was analyzed using FACSDiva software (BD Biosciences).

### Mice

VillinCre; Neurog3^{flox/flox} mice4 and B6.Cg-Gt(ROSA)26Sor^{tm9(CAG-tdTomato)Hze}/J (tdTomato)39 mice were genotyped as previously described. Mice were housed in a specific pathogen free barrier facility in accordance with NIH Guidelines for the Care and Use of Laboratory Animals. All experiments were approved by the Cincinnati Children's Hospital Research Foundation Institutional Animal Care and Use Committee (IACUC2019-0006) and carried out using standard procedures. Mice were maintained on a 12-hour light/dark cycle and had ad libitum access to standard chow and water.

VillinCre;Neurog3^{flox/flox} mice⁴ and their littermates were weighed, genotyped and visually examined for liquid feces daily beginning at postnatal day 10. Applicant established a diarrhea score, with 3 representing wet, yellow feces that smeared the perianal fur, and 0 representing normal, dry, brown, well-defined pellets. Mutant mice which suffered from diarrhea score 3 were included in the rescue experiment. 10 μg PYY (Phoenix Pharmaceuticals) was diluted in water and added to 20 μl DPP4 inhibitor (Millipore) to a final volume of 100 μl per mouse. Mice were injected intraperitoneally with this cocktail within 2 hours of the onset of the dark cycle (7 pm) daily until analysis at postnatal day 28-35. Mice were given access to solid chow on the floor of the cage beginning at postnatal day 10 and weaned at postnatal day 21.

NSG mice hosting HIOs were treated with 25 μg PYY (Phoenix Pharmaceuticals) diluted in water to 100 μL by intraperitoneal injection. Mice were treated daily for a minimum of 10 days after HIOs had been maturing for 8 weeks, then dissected and analyzed.

### Statistics

Data is presented as the mean+s.e.m. unless otherwise indicated. Significance was determined using unpaired, two-tailed Student's t-test, with p>0.05 not significant; *p<0.05, **p<0.01, ***p<0.001.

### REFERENCES

1 Wang, J. et al. Mutant neurogenin-3 in congenital malabsorptive diarrhea. New England Journal of Medicine 355, 270-280 (2006).

2 Gribble, F. M. & Reimann, F Function and mechanisms of enteroendocrine cells and gut hormones in metabolism. Nat Rev Endocrinol 15, 226-237, doi:10.1038/s41574-019-0168-8 (2019).

3 Cox, H. M. Neuroendocrine peptide mechanisms controlling intestinal epithelial function. Current Opinion in Pharmacology 31, 50-56 (2016).

4 Mellitzer, G. et al. Loss of enteroendocrine cells in mice alters lipid absorption and glucose homeostasis and impairs postnatal survival. The Journal of clinical investigation 120, 1708-1721 (2010).

5 Wright, E. M., Loo, D. D. F. & Hirayama, B. A. Biology of human sodium glucose transporters. Physiological reviews 91, 733-794 (2011).

6 Chen, M. et al. Gene ablation for PEPT1 in mice abolishes the effects of dipeptides on small intestinal fluid absorption, short-circuit current, and intracellular pH. American Journal of Physiology-Gastrointestinal and Liver Physiology 299, G265-G274 (2010).

7 Wright, E. M., Hirsch, J. R., Loo, D. D. & Zampighi, G. A.

Regulation of Na+/glucose cotransporters. Journal of Experimental Biology 200, 287-293 (1997).

8 Thwaites, D. T. et al. H+/dipeptide absorption across the human intestinal epithelium is controlled indirectly via a functional Na+/H+ exchanger. Gastroenterology 122, 1322-1333 (2002).

9 Burleigh, D. E. & Banks, M. R. Stimulation of intestinal secretion by vasoactive intestinal peptide and cholera toxin. Autonomic Neuroscience 133, 64-75, doi:https://doi.org/10.1016/j.autneu.2006.08.004 (2007).

10 Yun, C. H. C. et al. cAMP-mediated inhibition of the epithelial brush border Na+/H+ exchanger, NHE3, requires an associated regulatory protein. Proceedings of the National Academy of Sciences 94, 3010-3015 (1997).

11 Hyland, N. P., Sjoberg, F., Tough, I. R., Herzog, H. & Cox, H. M. Functional consequences of neuropeptide Y Y 2 receptor knockout and Y2 antagonism in mouse and human colonic tissues. Br J Pharmacol 139, 863-871, doi:10.1038/sj.bjp.0705298 (2003).

12 Cox, H. M. et al. Peptide YY is critical for acylethanolamine receptor Gpr119-induced activation of gastrointestinal mucosal responses. Cell Metab 11, 532-542, doi:10.1016/j.cmet.2010.04.014 (2010).

13 Tough, I. R. et al. Endogenous peptide YY and neuropeptide Y inhibit colonic ion transport, contractility and transit differentially via Y1 and Y2 receptors. British journal of pharmacology 164, 471-484 (2011).

14 Moodaley, R., Smith, D. M., Tough, I. R., Schindler, M. & Cox, H. M. Agonism of free fatty acid receptors 1 and 4 generates peptide YY-mediated inhibitory responses in mouse colon. British journal of pharmacology 174, 4508-4522 (2017).

15 Spence, J. R. et al. Directed differentiation of human pluripotent stem cells into intestinal tissue in vitro. Nature 470, 105 (2011).

16 Watson, C. L. et al. An in vivo model of human small intestine using pluripotent stem cells. Nature medicine 20, 1310 (2014).

17 McGrath, P. S., Watson, C. L., Ingram, C., Helmrath, M. A. & Wells, J. M. The Basic Helix-Loop-Helix Transcription Factor NEUROG3 Is Required for Development of the Human Endocrine Pancreas. Diabetes 64, 2497-2505, doi:10.2337/db14-1412 (2015).

18 Jenny, M. et al. Neurogenin3 is differentially required for endocrine cell fate specification in the intestinal and gastric epithelium. Embo j 21, 6338-6347 (2002).

19 Zhang, X. et al. A Comprehensive Structure-Function Study of Neurogenin3 Disease-Causing Alleles during Human Pancreas and Intestinal Organoid Development. Developmental Cell, doi:https://doi.org/10.1016/j.devce1.2019.05.017 (2019).

20 Bohorquez, D. V. et al. An enteroendocrine cell-enteric glia connection revealed by 3D electron microscopy. PLoS One 9, e89881, doi:10.1371/journal.pone.0089881 (2014).

21 Kaelberer, M. M. et al. A gut-brain neural circuit for nutrient sensory transduction. Science 361, eaat5236 (2018).

22 Bohorquez, D. V. et al. Neuroepithelial circuit formed by innervation of sensory enteroendocrine cells. J Clin Invest 125, 782-786, doi:10.1172/jci78361 (2015).

23 Panaro, B. L. et al. The melanocortin-4 receptor is expressed in enteroendocrine L cells and regulates the release of peptide YY and glucagon-like peptide 1 in vivo. Cell metabolism 20, 1018-1029 (2014).

24 Egerod, K. L. et al. Profiling of G protein-coupled receptors in vagal afferents reveals novel gut-to-brain sensing mechanisms. Mol Metab 12, 62-75, doi:10.1016/j.molmet.2018.03.016 (2018).

25 McCauley, H. A. Enteroendocrine regulation of nutrient absorption. Journal of Nutrition, doi:nxz191 (2019).

26 Dekkers, J. F. et al. A functional CFTR assay using primary cystic fibrosis intestinal organoids. Nature Medicine 19, 939, doi:10.1038/nm.3201.

27 Foulke-Abel, J. et al. Human Enteroids as a Model of Upper Small Intestinal Ion Transport Physiology and Pathophysiology. Gastroenterology 150, 638-649 e638 (2016).

28 Clarke, L. L. A guide to Ussing chamber studies of mouse intestine. Am J Physiol Gastrointest Liver Physiol 296, G1151-1166, doi:10.1152/ajpgi.90649.2008 (2009).

29 Anderson, C. M. H., Mendoza, M. E., Kennedy, D. J., Raldua, D. & Thwaites, D. T. Inhibition of intestinal dipeptide transport by the neuropeptide VIP is an anti-absorptive effect via the VPAC1 receptor in a human enterocyte-like cell line (Caco-2). British Journal of Pharmacology 138, 564-573, doi:10.1038/sj.bjp.0705049 (2003).

30 Egerod, K. L. et al. A major lineage of enteroendocrine cells coexpress CCK, secretin, GIP, GLP-1, PYY, and neurotensin but not somatostatin. Endocrinology 153, 5782-5795 (2012).

31 Mentlein, R., Dahms, P., Grandt, D. & Kruger, R. Proteolytic processing of neuropeptide Y and peptide YY by dipeptidyl peptidase IV. Regul Pept 49, 133-144 (1993).

32 Batterham, R. L. et al. Gut hormone PYY3-36 physiologically inhibits food intake.

33 Ouchi, R. et al. Modeling Steatohepatitis in Humans with Pluripotent Stem Cell-Derived Organoids. Cell Metabolism, doi:https://doi.org/10.1016/j.cmet.2019.05.007 (2019).

All percentages and ratios are calculated by weight unless otherwise indicated.

All percentages and ratios are calculated based on the total composition unless otherwise indicated.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “20 mm” is intended to mean “about 20 mm.”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications may be made without departing from the spirit and scope of the invention. It is therefore intended to cover in the appended claims all such changes and modifications that are within the scope of this invention.

1.-31. (canceled)

32. An intestinal organoid comprising a null mutation in NEUROG3 and lacking enteroendocrine cells (EECs), wherein the intestinal organoid is differentiated from pluripotent stem cells and do not express Chromogranin A (CHGA).

33. A method comprising contacting the intestinal organoid of claim 32 with a compound and assessing the effect of the compound on the intestinal organoid by immunofluorescence, flow cytometry, qPCR, a swelling assay, NHE3 activity assay, electrophysiology assay with an Ussing chamber, glucose uptake assay, or intracellular pH assay.


SEQUENCE LISTING (8 sequences)

SEQ ID NO: 1 - DNA, 21 bp, Artificial Sequence
  PPIA (CPHA) FWD
cccaccgtgttcttcgacatt

SEQ ID NO: 2 - DNA, 22 bp, Artificial Sequence
  PPIA (CPHA) REV
ggacccgtatgctttaggatga

SEQ ID NO: 3 - DNA, 20 bp, Artificial Sequence
  CFTR FWD
ggcacccagagtagtaggtc

SEQ ID NO: 4 - DNA, 20 bp, Artificial Sequence
  CFTR REV
aggcgctgtctgtatccttt

SEQ ID NO: 5 - DNA, 20 bp, Artificial Sequence
  SLC9A3 (NHE3) FWD
gctggtcttcatctccgtgt

SEQ ID NO: 6 - DNA, 20 bp, Artificial Sequence
  SLC9A3 (NHE3) REV
ccagaggcttgatggtcagg

SEQ ID NO: 7 - protein, 36 aa, Artificial Sequence
  Human-analogous PYY peptide
YPIKPEAPGEDASPEELNRYYASLRHYLNLVTRQRY

SEQ ID NO: 8 - protein, 36 aa, Artificial Sequence
  Non-human PYY peptide
YPAKPEAPGEDASPEELSRYYASLRHYLNLVTRQRY